(19) United States
(12) Patent Application Publication
(10) Pub. No.: US 2003/0152628 A1
(43) Pub. Date: Aug. 14, 2003

(54) Dual release formulation comprising levodopa ethyl ester and a decarboxylase inhibitor in an immediate-release layer with levodopa ethyl ester in a controlled release core

(76) Inventors: Daniela Licht, Ramat Ilan Givat Shmuel (IL); Shulamit Patashnik, Reut (IL); Ezmira Naftali, Rosh-Ha'ayin (IL); Naim Sayag, Yuvalim (IL); Adrian Gilbert, Ra'anana (IL); Sasson Cohen, Tel Aviv (IL); Corinne Zollmann, Kochav Yair (IL)

Correspondence Address: Cooper & Dunham LLP, 1185 Avenue of the Americas, New York, NY, 10036, US

(21) Appl. No.: 10/194,507

(22) Filed: Jul. 12, 2002

Related U.S. Application Data
(60) Provisional application No. 60/346,719, filed on Jan. 7, 2002.
(60) Provisional application No. 60/305,179, filed on Jul. 12, 2001.

(51) Int. Cl. A61K 9/24

(52) U.S. Cl. 424/472

(57) ABSTRACT

A tablet which comprises

an inner core formulated for controlled release consisting essentially of a mixture of levodopa ethyl ester or a derivative or a pharmaceutically acceptable salt thereof, a carrier and an inner core excipient; and

an outer layer encapsulating the inner core and formulated for immediate release comprising a mixture of a decarboxylase inhibitor and levodopa ethyl ester or a derivative or a pharmaceutically acceptable salt thereof.

The subject

invention also encompasses a method of treating patients suffering from Parkinson's disease or related disorders by the administration of the pharmaceutical compositions of the subject invention. The subject invention also provides methods of manufacturing the tablets of the subject invention.

This application claims the benefit of U.S. Provisional Application No. 60/346,719, filed Jan. 7, 2002, and U.S. Provisional Application No. 60/305,179, filed Jul. 12, 2001, both of which are hereby incorporated by reference.

Throughout this application, various references are referenced by citations within parenthesis. These references, in their entireties, are hereby incorporated by reference to more fully describe the state of the art to which this invention pertains.

### FIELD OF THE INVENTION

This invention relates to the treatment of Parkinson's disease (PD) and related disorders with levodopa (L-DOPA) and a decarboxylase inhibitor.

### BACKGROUND OF THE INVENTION

Parkinsonian patients are routinely treated with a combination of levodopa (L-DOPA) and a decarboxylase inhibitor such as carbidopa or benserazide (see e.g., U.S. Pat. Nos. 6,218,566, 5,525,631 and 5,354,885). Unfortunately, after an initial period of satisfactory, smooth and stable clinical benefits from L-DOPA therapy lasting on the average 2-5 years, the condition of many patients deteriorate and they develop complex dose-related as well as unpredictable response fluctuations. The causes of the response fluctuations are probably multiple and complex, but pharmacokinetic problems (primarily faulty absorption of L-DOPA) may play a critical role. There is a correlation between the clinical fluctuations and the oscillations of L-DOPA plasma levels. Many of the problems are a result of the unfavorable pharmacokinetic properties of L-DOPA, i.e., very poor solubility, poor bioavailability and short half-life in vivo.

A typical problem for Parkinsonian patients is the “on-off” oscillations in which daily motor activity is dominated by remarkable swings between “off” hours, when they are severely incapacitated, rigid, unable to move and sometimes to speak or swallow, to “on” periods where they are responsive to L-DOPA and can, more or less, perform. The current treatments (apomorphine, lisuride) used to treat patients in the “off” period are unsatisfactory.

Various procedures have been attempted to remedy this situation. In some cases, direct instillation of a slurry of levodopa through a duodenal tube has given rapid relief from the “off” state (Durlan R. et al. (1986), Ann. Neurol.20: 262-265 and Cedarbaum et al. (1990), Neurology 40:878-995). In another approach, oral dosing with a dilute aqueous solution of levodopa appeared to be effective (Kurth M. C. et al. (1993), Neurology 43:1036-1039). However, neither of these measures are practical enough to allow self-medication when urgently needed. When rapid relief is needed, the more common procedure is to recommend the patients to crush the levodopa tablet before intake so as to minimize the time required for its disintegration in the gastrointestinal (GI) tract. The efficacy of this procedure has never been demonstrated.

A major problem in long-term treatment of PD with chronic intermittent levodopa therapy is fluctuating motor response—the “on-off” phenomenon and the increasingly frequent appearance of dyskinesia. There is some evidence that these often quite disturbing variations in drug response are due, in part, to fluctuations in drug plasma concentration which is responsible for the early (but temporary) severe dyskinetic bouts and the quickly dropping plasma levels may well be the cause of premature “end of dose” aggravations of the motor disability.

An important approach to the treatment of those phenomena is the attempt to prolong the duration of levodopa in the plasma with the use of sustained, controlled-release (CR) preparations. Such a dosage form is currently available under the brand name SINEMET CR® (Merck Sharpe & Dohme), which is a compressed tablet containing controlled release carbidopa-levodopa. This formulation produces a constant rise in plasma levodopa level that is sustained for 3 to 4 hours, which is significantly longer than that obtained with immediate-release carbidopa-levodopa preparations. An initial absorption phase is lacking, but a gradual build-up in the absorption profile occurs. Peak levels are not recorded until after 2 hours. SINEMET CR® is available in two forms: 1) SINEMET CR 50-200®, containing 50 mg carbidopa and 200 mg levodopa; and 2) SINEMET CR 25-100®, containing 25 mg carbidopa and 100 mg levodopa.

Controlled-release carbidopa-levodopa, such as SINEMET CR®, has approximately 30% less bioavailability compared to immediate-release preparations, such as SINEMET®. Considerable inter-subject variation has been observed in levodopa absorption. Peak levodopa plasma levels following administration of controlled-release carbidopa-levodopa are lower than those found with immediate-release preparations.

A number of open-label studies of controlled-release carbidopa-levodopa in PD patients with motor fluctuations have demonstrated a significant reduction in “off” time and improvement in clinical disability scores. Goetz et al. compared the controlled-release formulation of carbidopa-levodopa to the immediate-release preparations in an open-label trial in 20 PD patients with “wearing-off” phenomena and found increased “on” time after 4 to 6 weeks of therapy ((1987), Neurology 37: 875-878). In 9 patients followed for 3 months, the “on” time” without chorea remained significantly increased. In another open label study of 17 PD patients with severe fluctuations, controlled-release carbidopa-levodopa, when compared with the immediate-release formulation, caused a reduction in the number of “off” periods and a slight increase in “on” time. Some patients required additional immediate-release carbidopa-levodopa.

Friedman and Lannon noted that “wearing-off” but not “on-off” phenomena improved in 19 patients in a 1-year open-label study ((1989), Clinical Neuropharmacol.12: 220-223). In 20 PD patients with motor fluctuations treated with controlled-release carbidopa-levodopa for one year, Rondot et al. reported an improvement in clinical scores and a 63% prolongation of “on” periods ((1989), Neurology. 39(suppl 2): 74-77).

In a 52-week open-label trial of 20 patients, it was noted that with controlled-release carbidopa-levodopa, fluctuations became less troublesome, but did not disappear (Aarli, J. A., Gilhus, N. E. (1989), Neurology. 39 (suppl 2): 82-85). In an open-label trial involving eight PD patients with fluctuations given controlled-release carbidopa-levodopa for 36 to 39 months, five patients experienced an increase in daily “on” time when compared with baseline (Rodnitzki, R. L. et al. (1989), Neurology. 39 (suppl 2): 92-95)

Pahwa et al. converted 158 patients from immediate-release to controlled-release carbidopa-levodopa ((1993), Neurology. 43: 677-681) and found that the “off” time decreased significantly. 73% of the patients preferred the controlled-release preparation; Pahwa et al. concluded that controlled-release carbidopa-levodopa was particularly effective in decreasing motor fluctuations in PD patients with mild-to-moderate disease. In a study of 17 patients with motor fluctuations, immediate-release and controlled-release carbidopa-levodopa were compared over several doses during one day. During treatment with the controlled-release preparation, total “on” time was increased, and the number of “off” episodes was reduced.

It appears that controlled-release carbidopa-levodopa preparations provide a more stable and constant levodopa plasma level than immediate-release formulations. Controlled-release preparations are efficacious in the treatment of motor fluctuations in PD, have longer duration of action for each dose, cause a decrease in dose failures, a reduction in early morning dystonia and a decrease in nocturnal awakenings.

However, controlled-release preparations also cause a slower or delayed onset of effect in some PD patients, which is related to the slow build-up of plasma levels of levodopa in the first dose. Therefore, some patients require an immediate-release preparation before taking the controlled-release preparation, especially for the first morning dose.

Rubin (U.S. Pat. No. 6,238,699 B1) discloses a pharmaceutical composition containing carbidopa and levodopa in immediate and controlled release compartments. Rubin teaches that his compositions may fall into any one of the following types: 1) a compressed inner tablet core onto which an outer tablet core is compressed (dual compression); 2) a capsule or compressed tablet containing pellets; or 3) a layer tablet comprising two or more layers (sandwich). However, Rubin does not describe how to obtain an effective formulation with any agent other than levodopa. Thus, Rubin does not teach how to formulate a tablet having two drugs with very different solubilities (Table 1).

As an alternative to levodopa, Chiesi et al. disclose pharmaceutical compositions comprising controlled release and immediate release formulations of levodopa methyl ester and carbidopa (WO 99/17745). Chiesi et al. suggest the prepartation of their pharmaceutical compositions as 3-layer monolithic tablets (sandwiches). Chiesi et al. provide no guidance concerning how to formulate compositions other than composition containing levodopa methyl ester. In one formulation, the slow release layer contains levodopa methyl ester, but not carbidopa, while the remaining layers employ both levodopa methyl ester and carbidopa. However, a significant detriment to the utility of the compositions of Chiesi et al. is that levodopa methyl ester is metabolized into L-DOPA and methanol, of which methanol is toxic (U.S. Pat. No. 5,354,885).

Another replacement for L-DOPA is levodopa ethyl ester (LDEE). LDEE increases the bioavailability of L-DOPA due to its greater solubility (U.S. Pat. No. 5,354,885, Milman et al.). LDEE was incorporated into pharmaceutical compositions by Cohen et al. (U.S. Pat. No. 5,840,756). Although Cohen et al. state that their pharmaceutical compositions provide “a burst of levodopa followed by the maintenance of a sustained level of levodopa” (from the metabolism of levodopa ethyl ester), the compositions are only controlled release compositions (see Example 2a). They disclose that their compositions may be formulated as single compression tablets and may contain a decarboxylase inhibitor.

Levin (WO 00/27385) combined levodopa ethyl ester and a decarboxylase inhibitor, carbidopa, in pharmaceutical compositions. However, in contrast to Cohen et al., the pharmaceutical compositions disclosed by Levin are solely immediate release formulations.

Thus, there is a need for an LDEE pharmaceutical composition with a decarboxylase inhibitor that will increase the bioavailability of levodopa. Such a pharmaceutical composition needs to dissolve rapidly in a patient requiring levodopa therapy, and at the same time, provide a sustained therapeutic level of levodopa in the patient, have good patient compliance and be easy to manufacture.

### SUMMARY OF THE INVENTION

The subject invention provides a tablet which comprises

an inner core formulated for controlled release consisting essentially of a mixture of levodopa ethyl ester or

a derivative or a pharmaceutically acceptable salt thereof, a carrier and an inner core excipient component; and

an outer layer encapsulating the inner core and formulated for immediate release comprising a mixture of a decarboxylase inhibitor and levodopa ethyl ester or a derivative or a pharmaceutically acceptable salt thereof.

The subject invention further provides a tablet which comprises an inner core formulated for controlled release comprising

(a) a pre-mixture of levodopa ethyl ester or a derivative or a pharmaceutically acceptable salt thereof and a carrier, and

(b) at least one inner core excipient component; and

an outer layer encapsulating the inner core and formulated for immediate release comprising a mixture of carbidopa and levodopa ethyl ester or a derivative or a pharmaceutically acceptable salt thereof.

The subject invention also provides a tablet which comprises an inner core formulated for controlled release comprising a mixture of

(a) from about 4 mg up to about 400 mg levodopa ethyl ester or a derivative or a pharmaceutically acceptable salt thereof, and

(b) an inner core excipient component;

and an outer layer encapsulating the inner core and formulated for immediate release comprising a mixture of

(i) a granulated admixture of from about 1 mg up to about 75 mg carbidopa and at least one excipient; and

(ii) from about 5 mg up to about 300 mg levodopa ethyl ester or a derivative or a pharmaceutically acceptable salt thereof, and

(iii) an outer layer excipient component.

The subject invention further provides a method of treating a subject suffering from a disease selected from the group consisting of Parkinson's disease, senile dementia, dementia of the Alzheimer's type, a memory disorder, depression, hyperactive syndrome, an affective illness, a neurodegenerative disease, a neurotoxic injury, brain ischemia, a head trauma injury, a spinal trauma injury, schizophrenia, an attention deficit disorder, multiple sclerosis, withdrawal symptoms, epilepsy, convulsions and seizures, which comprises administering to the subject the tablet of the subject invention in an amount effective to treat the disease.

Additionally, the subject invention provides a method of inducing in a human subject a therapeutically effective blood plasma level of levodopa comprising administering to the human subject

a controlled release formulation of levodopa ethyl ester, and

an immediate release formulation of levodopa ethyl ester,

wherein the therapeutically effective blood plasma level is at least 1000 ng of levodopa per ml of blood plasma within 50 minutes after the administration and no less than 100 ng of levodopa per ml of blood plasma at 6 hours after the administration.

The subject invention also provides a method of inducing in a human subject a therapeutically effective blood plasma level of levodopa and carbidopa comprising administering to the human subject

a controlled release formulation of levodopa ethyl ester, and

an immediate release formulation of levodopa ethyl ester, in admixture with carbidopa,

wherein the therapeutically effective blood plasma level is at least 1000 ng of levodopa and 100 ng of carbidopa per ml of blood plasma within 50 minutes after the administration and no less than 100 ng of levodopa and 40 ng of carbidopa per ml of blood plasma at 6 hours after the administration.

The subject invention further provides methods of manufacturing the tablets of the subject invention.

### BRIEF DESCRIPTION OF THE FIGURES

FIG. 1: The pH dependence of the dual release levodopa ethyl ester/carbidopa formulation of the subject invention. The rate of dissolution of LDEE is independent of the pH.

FIG. 2: The pH dependence of the commercial levodopa/carbidopa controlled release formulation (Sinemet CR®). The rate of dissolution of levodopa varies with increasing pH.

FIG. 3: Plasma concentration of levodopa following administration of a carbidopa/LDEE tablet of the subject invention.

FIG. 4: Plasma concentration of carbidopa following administration of a carbidopa/LDEE tablet of the subject invention.

### DETAILED DESCRIPTION OF THE INVENTION

The subject invention provides a tablet which comprises

an inner core formulated for controlled release consisting essentially of a mixture of levodopa ethyl ester or a derivative or a pharmaceutically acceptable salt thereof, a carrier and an inner core excipient component; and

an outer layer encapsulating the inner core and formulated for immediate release comprising a mixture of a decarboxylase inhibitor and levodopa ethyl ester or a derivative or a pharmaceutically acceptable salt thereof.

As used herein, a “derivative of levodopa ethyl ester” is a compound that has substantially the same effect as levodopa ethyl ester in the treatment of Parkinson's disease and related disorders. Derivatives of levodopa ethyl ester includes compounds having structures such as those disclosed in U.S. Pat. No. 4,873,263.

A pharmaceutically acceptable salt of levodopa ethyl ester is any pharmaceutically acceptable salt of levodopa ethyl ester, e.g., the hydrochloride salt, the octanoate salt, the myristate salt, the succinate salt, the succinate dihydrate salt, the fumarate salt, the fumarate dihydrate salt, the acetate salt, the mesylate salt, the esylate salt, the tartarate salt, the hydrogen tartarate salt, the benzoate salt, the phenylbutyrate salt, the phosphate salt, the citrate salt, the ascorbate salt, the mandelate salt, or the adipate salt of levodopa ethyl ester.

As used herein, the phrase, “controlled release” means the release of small increments over time, usually requiring several hours to achieve 100% dissolution. Controlled release formulations encompasses, for example, slow release, extended release and sustained release formulations.

As used herein, the phrase, “immediate release” indicates that the drug is allowed to dissolve in the gastrointestinal contents, with no intention of delaying or prolonging the dissolution or absorption of the drug (FDA Guidance for Industry SUPAC-MR: modified release oral dosage forms CDER, September, 1997). Immediate release formulations encompass, for example, rapid burst formulations.

In one embodiment, in the inner core, the levodopa ethyl ester or derivative or pharmaceutically acceptable salt thereof is in a pre-mixture with the carrier.

In another embodiment, the decarboxylase inhibitor is carbidopa.

In a further embodiment, the inner core formulated for controlled release consists essentially of a mixture of

(a) from about 4 mg up to about 400 mg levodopa ethyl ester or a derivative or a pharmaceutically acceptable salt thereof,

(b) a carrier, and

(c) an inner core excipient component; and

the outer layer encapsulating the inner core and formulated for immediate release comprises a mixture of:

(i) from above 0 mg up to about 200 mg carbidopa,

(ii) from about 5 mg up to about 300 mg levodopa ethyl ester or a derivative or a pharmaceutically acceptable salt thereof, and

(iii)an outer layer excipient component.

In yet another embodiment, components (a) and (b) are in a pre-mixture.

In an additional embodiment, in the outer layer, the carbidopa in (i) comprises granulated carbidopa.

In one embodiment, in the inner core, the levodopa ethyl ester or derivative or pharmaceutically acceptable salt thereof and the carrier are in a ratio of about 2:1 by weight.

In this application, the phrase “substantially the same as” with reference to comparison of rates of release of components of the inner core or of components from the outer layer of a tablet means that the rates of release of the compounds being compared are the same, or if the rates differ, they differ by less than 35% between or among the components being compared.

Non-limiting examples of a carrier (extended release agent) used in the subject invention (used for example for the controlled release) are cellulose acetate, glyceryl monostearate, zein, microcrystalline wax, hydroxymethylcellulose, hydroxyethylcellulose, hydroxypropylmethylcellulose, carboxyvinyl polymers, polyvinyl alcohols, glucans, scleroglucans, chitosans, mannans, galactomannans, amylose, alginic acid and salts and derivatives thereof, acrylates, methacrylates, acrylic/methacrylic copolymers, polyanhydrides, polyaminoacids, methyl vinyl ethers/maleic anhydride copolymers, carboxymethylcellulose and derivatives thereof, ethylcellulose, methylcellulose and cellulose derivatives in general, modified starch and polyesters, polyethylene oxide.

In a preferred embodiment, the carrier comprises a hydroxypropylmethylcellulose. In another preferred embodiment, the hydroxypropylmethylcellulose has an average molecular weight between about 10 kDa and about 1500 kDa. In a further preferred embodiment, the hydroxypropylmethylcellulose has 19%-24% methoxyl substituent and 7%-12% hydroxylproproxyl substituent. In an added embodiment, the hydroxypropylmethylcellulose has a particle size distribution such that about 100% of the hydroxypropylmethylcellulose passes through a 30 mesh screen. In one embodiment, the hydroxypropylmethylcellulose has a particle size distribution such that about 99% of the hydroxypropylmethylcellulose passes through a 40 mesh screen. In yet another embodiment, the hydroxypropylmethylcellulose has a particle size distribution such that 55%-95% of the hydroxypropylmethylcellulose passes through a 100 mesh screen. In a further embodiment, the hydroxypropylmethylcellulose has a particle size distribution such that 65%-85% of the hydroxypropylmethylcellulose passes through a 100 mesh screen. In an additional embodiment, the hydroxypropylmethylcellulose has a particle size distribution such that about 80% of the hydroxypropylmethylcellulose passes through a 100 mesh screen. In a further embodiment, the hydroxypropylmethylcellulose has a particle size distribution such that about 90% of the hydroxypropylmethylcellulose passes through a 100 mesh screen. In a preferred embodiment, the hydroxypropylmethylcellulose is a Methocel®, such as Methocel K100LVP® (also known as Methocel K100LV®) or Methocel K15MP® (also known as Methocel K15M®).

In one embodiment, the outer layer excipient component and/or the inner core excipient component comprises an excipient used as a binding agent. Non-limiting examples of a binding agent used in the subject invention (used for example for the granulate) are alginic acid, acia, carbomer, carboxymethylcellulose sodium, dextrin, ethylcellulose, gelatin, guar gum, hydrogenated vegetable oil, hydroxyethyl cellulose, hydroxypropylcellulose (e.g., Klucel®), hydroxypropylmethylcellulose, liquid glucose, magnesium aluminum silicate, maldodextrin, methylcellulose, polymethacrylates, povidone, pregelatinized starch, sodium alginate, starch, and zein. In a preferred embodiment, the excipient used as a binding agent comprises a hydroxypropylcellulose.

In an additional embodiment, the outer layer excipient component comprises an excipient used as a disintegrating agent. Non-limiting examples of a disintegrant used in the subject invention (used for example for the disintegration of immediate release tablet) are kaolin, starch, powdered sugar, sodium starch glycolate, crosscarmelose sodium, carboxymethyl cellulose, microcrystalline cellulose and sodium alginate. In a preferred embodiment, the excipient used as a disintegrating agent comprises a starch. In another preferred embodiment, the starch is a partially pregelatinized maize starch, such as Starch 1500®.

In yet another embodiment, the inner core excipient component and the outer layer excipient component each comprise an excipient useful as a flow agent and/or an excipient useful as a lubricant.

In one embodiment, the excipient useful as a flow agent comprises a micron-sized silica powder. A non-limiting example of a flow agent used in the subject invention (used for better flow of the mix for compression) is colloidal silicon dioxide or Syloid®, which is a preferred embodiment.

Non-limiting examples of a lubricant used in the subject invention (used for example for better compression properties) are talc, sodium stearyl fumarate, magnesium stearate, calcium stearate, hydrogenated castor oil, hydrogenated soybean oil and polyethylene glycol (PEG) or combinations thereof. In a preferred embodiment, the excipient useful as a lubricant comprises magnesium stearate. In another preferred embodiment, the excipient useful as a lubricant comprises sodium stearyl fumarate.

In a further embodiment, the inner core excipient component and the outer layer excipient component each comprise an excipient useful as a lubricant.

In another embodiment, the same excipient useful as a lubricant is present in both the inner core excipient component and the outer layer excipient component.

In an additional embodiment, the excipient useful as a lubricant present in the outer core excipient component comprises sodium stearyl fumarate.

In a further embodiment, the excipient useful as a lubricant present in the inner core excipient component comprises sodium stearyl fumarate.

In yet another embodiment, the inner core excipient component comprises a first excipient useful as a lubricant and a second excipient useful as a lubricant. In a preferred embodiment, the first excipient usesful as a lubricant is sodium stearyl fumarate and the second excipient useful as a lubricant is magnesium stearate.

In one embodiment, the inner core excipient component and/or the outer layer excipient component comprises an excipient useful as a filler. Fillers may be inorganic or organic materials, and may be soluble or insoluble. Non-limiting examples of a filler used in the subject invention (used for example for weight adjustment and for better compression) are corn starch, lactose, glucose, various natural gums, methylcellulose, carboxymethylcellulose, microcrystalline cellulose, calcium phosphate, calcium carbonate, calcium sulfate kaolin, sodium chloride, powdered cellulose, sucrose, mannitol and starch. In a preferred embodiment, the excipient useful as a filler comprises a microcrystalline cellulose. In a more preferred embodiment, the microcrystalline cellulose has an average particle size between about 50 and about 90 microns. In a preferred embodiment, the microcrystalline cellulose is Avicel PH 101®, which has an average particle size of 50 microns. In another preferred embodiment, the microcrystalline cellulose is Avicel PH 112®, which has an average particle size of 90 microns.

In another embodiment, in the inner core

the levodopa ethyl ester or derivative or pharmaceutically acceptable salt thereof in (a) is present in an amount from about 10 mg up to about 400 mg;

and wherein in the outer layer,

the granulated carbidopa in (i) comprises from above 0 mg to about 75 mg carbidopa, and

the levodopa ethyl ester or derivative or pharmaceutically acceptable salt thereof in (ii) is present in an amount from about 10 mg up to about 250 mg.

In a further embodiment, in the inner core

the levodopa ethyl ester or derivative or pharmaceutically acceptable salt thereof is present in an amount from about 50 mg up to about 400 mg;

and wherein in the outer layer

the granulated carbidopa comprises from about 10 mg up to about 50 mg carbidopa, and

the amount of the levodopa ethyl ester or the derivative or pharmaceutically acceptable salt thereof is from about 50 mg up to about 200 mg.

In yet another embodiment, in the inner core

the levodopa ethyl ester or derivative or pharmaceutically acceptable salt thereof is present in an amount from about 19 mg up to about 228 mg;

and wherein in the outer layer

the granulated carbidopa comprises from about 4.2 mg up to about 75 mg carbidopa, and

the levodopa ethyl ester or derivative or pharmaceutically acceptable salt thereof is present in an amount from about 19 mg up to about 228 mg.

In one embodiment, above 5% of the total levodopa ethyl ester or derivative or pharmaceutically acceptable salt thereof present in the tablet is in the outer layer.

In another embodiment, above 10% of the total levodopa ethyl ester or derivative or pharmaceutically acceptable salt thereof present in the tablet is in the outer layer.

In an additional embodiment, above 30% of the total levodopa ethyl ester or derivative or pharmaceutically acceptable salt thereof present in the tablet is in the outer layer.

In one embodiment, above 50% of the total levodopa ethyl ester or derivative or pharmaceutically acceptable salt thereof present in the tablet is in the outer layer.

In a further embodiment, above 70% of the total levodopa ethyl ester or derivative or pharmaceutically acceptable salt thereof present in the tablet is in the outer layer.

In one embodiment, the total tablet comprises about 228.0 mg levodopa ethyl ester or derivative or pharmaceutically acceptable salt thereof, and about 50.0 mg carbidopa.

In an added embodiment, the total tablet comprises about 114.0 mg levodopa ethyl ester or derivative or pharmaceutically acceptable salt thereof, and about 25.0 mg carbidopa.

In yet another embodiment, the total tablet comprises about 57.0 mg levodopa ethyl ester or derivative or pharmaceutically acceptable salt thereof, and about 12.5 mg carbidopa.

In a further embodiment, in the inner core

the 4 mg up to about 400 mg levodopa ethyl ester or derivative or pharmaceutically acceptable salt thereof is present,

the carrier comprises from about 2.5 mg up to about 245 mg hydroxypropylmethylcellulose, and

the inner core excipient component comprises from 0 up to about 150 mg of a microcrystalline cellulose, from about 1 mg to about 10 mg of a micron-sized silica and from about 1 mg to about 30 mg sodium stearyl fumarate and about 1 mg to about 10 mg magnesium stearate,

and wherein in the outer layer

the granulated carbidopa is present in an amount from about 1 mg up to about 75 mg, and is present in a granulated admixture with 0 mg up to about 300 mg of a microcrystalline cellulose, from above 0 mg up to about 300 mg of a partially pregelatinized maize starch and from above 0 mg up to about 50 mg of a hydroxypropylcellulose,

from about 5 mg up to about 300 mg levodopa ethyl ester is present, and

the outer layer excipient component comprises above 0 mg up to about 300 mg of a microcrystalline cellulose, above 0 up to about 300 mg of a partially pregelatinized maize starch above 0 mg up to about 50 mg of a micron-sized silica and from above 0 up to about 30 mg sodium stearyl fumarate.

In one embodiment, in the inner core

about 114 mg levodopa ethyl ester is present;

the carrier comprises about 50 mg of the hydroxypropylmethylcellulose; and

the inner core excipient component comprises about 85 mg of a microcrystalline cellulose, about 2.7 mg of a micron-sized silica, about 5 mg of sodium stearyl fumarate and about 2.5 mg magnesium stearate,

and wherein in the outer layer

about 54 mg granulated carbidopa is present in a granulated admixture with about 40 mg of a microcrystalline cellulose, about 34 mg of a partially pregelatinized maize starch, and about 12 mg of a hydroxypropyl cellulose,

about 114 mg levodopa ethyl ester is present, and

the outer layer excipient component comprises about 132 mg of a microcrystalline cellulose, about 4 mg of a partially pregelatinized maize starch, about 3 mg of a micron-sized silica, and about 7.5 mg sodium stearyl fumarate.

In still another embodiment, in the inner core

about 114 mg levodopa ethyl ester is present;

the carrier comprises about 140 mg of a hydroxypropylmethylcellulose; and

the inner core excipient component comprises about 2.7 mg of a micron-sized silica, about 5 mg of sodium stearyl fumarate and about 2.5 mg magnesium stearate,

and wherein in the outer layer

about 54 mg granulated carbidopa is present in a granulated admixture with about 40 mg of a microcrystalline cellulose, about 34 mg of a partially pregelatinized maize starch, and about 12 mg of a hydroxypropyl cellulose,

about 114 mg levodopa ethyl ester is present, and

the outer layer excipient component comprises about 132 mg of a microcrystalline cellulose, about 4 mg of a partially pregelatinized maize starch, about 3 mg of a micron-sized silica, and about 7.5 mg sodium stearyl fumarate.

In an additional embodiment, in the inner core

about 152 mg levodopa ethyl ester is present,

the carrier comprises about 57 mg hydroxypropylmethylcellulose; and

the inner core excipient component comprises about 41 mg of a microcrystalline cellulose, about 2.7 mg of a micron-sized silica, about 5 mg sodium stearyl fumarate and about 2.5 mg magnesium stearate,

and wherein in the outer layer

the granulated carbidopa is present in an amount of about 54 mg, and is present in a granulated admixture with about 40 mg of a microcrystalline cellulose, about 34 mg of a partially pregelatinized maize starch and about 12 mg of a hydroxypropylcellulose,

about 76 mg levodopa ethyl ester is present, and

the outer layer excipient component comprises about 170 mg of a microcrystalline cellulose, about 4 mg of a partially pregelatinized maize starch, about 3 mg of a micron-sized silica and about 7.5 mg sodium stearyl fumarate.

In a further embodiment, in the inner core

about 152 mg levodopa ethyl ester is present,

the carrier comprises about 40 mg hydroxypropylmethylcellulose, and

the inner core excipient component comprises about 54 mg of a microcrystalline cellulose, about 2.7 mg of a micron-sized silica, about 5 mg sodium stearyl fumarate and about 2.5 mg magnesium stearate;

and wherein in the outer layer

the granulated carbidopa is present in an amount of about 54 mg, and is present in a granulated admixture with about 40 mg of a microcrystalline cellulose, about 34 mg of a partially pregelatinized maize starch and about 12 mg of a hydroxypropylcellulose,

about 76 mg levodopa ethyl ester is present, and

the outer layer excipient component comprises about 170 mg of a microcrystalline cellulose, about 4 mg of a partially pregelatinized maize starch, about 3 mg of a micron-sized silica and about 7.5 mg sodium stearyl fumarate.

In one embodiment, in the inner core

about 182 mg levodopa ethyl ester is present,

the carrier comprises about 40 mg hydroxypropylmethylcellulose, and

the inner core excipient component comprises about 23 mg of a microcrystalline cellulose, about 2.7 mg of a micron-sized silica, about 5 mg sodium stearyl fumarate and about 2.5 mg magnesium stearate;

and wherein in the outer layer

the granulated carbidopa is present in an amount of about 54 mg, and is present in a granulated admixture with about 40 mg of a microcrystalline cellulose, about 34 mg of a partially pregelatinized maize starch and about 12 mg of a hydroxypropylcellulose,

about 46 mg levodopa ethyl ester is present, and

the outer layer excipient component comprises about 200 mg of a microcrystalline cellulose, about 4 mg of a partially pregelatinized maize starch, about 3 mg of a micron-sized silica and about 7.5 mg sodium stearyl fumarate.

The subject invention also provides a tablet which comprises

an inner core formulated for controlled release comprising

(a) a pre-mixture of levodopa ethyl ester or a derivative or a pharmaceutically acceptable salt thereof and a carrier, and

(b) at least one inner core excipient component; and

an outer layer encapsulating the inner core and formulated for immediate release comprising a mixture of carbidopa and levodopa ethyl ester or a derivative or a pharmaceutically acceptable salt thereof.

The particular elements and amounts of this tablet are as described above for analogous elements and amounts.

In one embodiment, in the inner core, the levodopa ethyl ester or derivative or pharmaceutically acceptable salt thereof and the carrier are in a ratio of about 2:1 by weight.

In a further embodiment, in the inner core, the levodopa ethyl ester or derivative or pharmaceutically acceptable salt thereof and the carrier are in a ratio of about 3:1 by weight.

The subject invention additionally provides a tablet which comprises

an inner core formulated for controlled release comprising a mixture of

(a) from about 4 mg up to about 400 mg levodopa ethyl ester or a derivative or a pharmaceutically acceptable salt thereof, and

(b) an inner core excipient component;

and an outer layer encapsulating the inner core and formulated for immediate release comprising a mixture of

(i) a granulated admixture of from about 1 mg up to about 75 mg carbidopa and at least one excipient; and

(ii) from about 5 mg up to about 300 mg levodopa ethyl ester or a derivative or a pharmaceutically acceptable salt thereof, and

(iii)an outer layer excipient component.

The particular elements and amounts of this tablet are as described above for analogous elements and amounts.

The subject invention further provides a process for manufacturing the tablet of the subject invention, comprising

(A) mixing the levodopa ethyl ester or derivative or pharmaceutically acceptable salt thereof with the carrier and the inner core excipient component;

(B) compressing the mixture from step (A) to form an inner core;

(C) separately mixing the decarboxylase inhibitor with the levodopa ethyl ester or derivative or pharmaceutically acceptable salt thereof and an outer layer excipient component; and

(D) compressing the mixture of step (C) over the inner core formed in step (B) to form an outer layer encapsulating the inner core so as to thereby manufacture the tablet.

In one embodiment, in step (A), the levodopa ethyl ester or derivative or pharmaceutically acceptable salt thereof is present in an amount by weight equal to or greater than an amount by weight of the carrier and/or in step (C), the carbidopa and the levodopa ethyl ester or derivative or pharmaceutically acceptable salt thereof are in a ratio by weight of carbidopa to levodopa ethyl ester from about 0.01:1 up to about 1.5:1.

In another embodiment, the process comprises

(A) preparing a pre-mixture of the levodopa ethyl ester or derivative or pharmaceutically acceptable salt thereof and the carrier;

(B) mixing the pre-mixture from step (A) with the inner core excipient component;

(C) compressing the mixture from step (B) to form an inner core;

(D) separately mixing the decarboxylase inhibitor with an outer layer excipient component and the levodopa ethyl ester or derivative or pharmaceutically acceptable salt thereof; and

(E) compressing the mixture of step (D) over the inner core formed in step (C) to form an outer layer encapsulating the inner core so as to thereby manufacture the tablet.

In an added embodiment, in step (A), the levodopa ethyl ester or derivative or pharmaceutically acceptable salt thereof is present in an amount by weight equal to or greater than an amount by weight of the carrier and/or in step (B), the levodopa ethyl ester or derivative or salt thereof and the inner core excipient component are in a ratio by weight from about 0.3:1 up to about 5.5:1, and/or in step (D), the carbidopa and the levodopa ethyl ester or derivative or pharmaceutically acceptable salt thereof are in a ratio by weight of from about 0.01:1 up to about 1.5:1.

In yet another embodiment, the process comprises

(A) mixing from about 4 mg up to about 400 mg levodopa ethyl ester or a derivative or a pharmaceutically acceptable salt thereof with a carrier and an inner core excipient component;

(B) compressing the mixture from step (A) to form the inner core;

(C) separately mixing above 0 mg up to about 75 mg carbidopa with about 5 mg up to about 300 mg levodopa ethyl ester or a derivative or a pharmaceutically acceptable salt thereof and an outer layer excipient component; and

(D) compressing the mixture of step (C) over the inner core formed in step (B) to form an outer layer encapsulating the inner core so as to thereby manufacture the tablet.

In a further embodiment, the levodopa ethyl ester or derivative or pharmaceutically acceptable salt thereof is present in an amount by weight equal to or greater than an amount by weight of the carrier and/or in step (C), the carbidopa and levodopa ethyl ester or derivative or pharmaceutically acceptable salt thereof are in a ratio by weight of from about 0.01:1 up to about 1.5:1.

In one embodiment, in the outer layer, the decarboxylase inhibitor comprises carbidopa.

In a further embodiment, the carbidopa comprises granulated carbidopa.

In still another embodiment, the process comprises

(A) mixing about 4 mg up to about 400 mg of levodopa ethyl ester or derivative or pharmaceutically acceptable salt thereof with a carrier;

(B) mixing an inner core excipient component with the mixture from step (A);

(C) compressing the mixture from step (B) to form the inner core;

(D) separately mixing 0 mg up to about 75 mg of carbidopa, about 5 mg up to about 300 mg of levodopa ethyl ester or derivative or pharmaceutically acceptable salt thereof and an outer layer excipient component; and

(E) compressing the mixture of step (D) over the inner core formed in step (C) to form an outer layer encapsulating the inner core so as to thereby manufacture the tablet.

In an additional embodiment, wherein in step (A), the levodopa ethyl ester or derivative or pharmaceutically acceptable salt thereof is present in an amount by weight equal to or greater than an amount by weight of the carrier and/or in step (B), the levodopa ethyl ester or derivative or pharmaceutically acceptable salt thereof and the inner core excipient component are in a ratio by weight from about 0.3:1 up to about 5.5:1, and/or in step (D), the carbidopa and the levodopa ethyl ester or derivative or pharmaceutically acceptable salt thereof are in a ratio by weight of from about 0.01:1 up to about 1.5:1.

In a further embodiment, the process comprises

(A) mixing from about 4 mg up to about 400 mg levodopa ethyl ester or a derivative or a pharmaceutically acceptable salt thereof with an inner core excipient component;

(B) compressing the mixture from step (A) to form the inner core;

(C) separately granulating from about 1 mg up to about 75 mg carbidopa with at least one excipient;

(D) mixing the granulated admixture from step (C) with about 5 mg up to about 300 mg levodopa ethyl ester or a derivative or a pharmaceutically acceptable salt thereof and an outer layer excipient component;

(E) compressing the mixture of step (D) over the inner core formed in step (B) to form an outer layer encapsulating the inner core so as to thereby manufacture the tablet.

The subject invention also provides a method of treating a subject suffering from a disease selected from the group consisting of Parkinson's disease, senile dementia, dementia of the Alzheimer's type, a memory disorder, depression, hyperactive syndrome, an affective illness, a neurodegenerative disease, a neurotoxic injury, brain ischemia, a head trauma injury, a spinal trauma injury, schizophrenia, an attention deficit disorder, multiple sclerosis, withdrawal symptoms, epilepsy, convulsions and seizures, which comprises administering to the subject the tablet of the subject invention in an amount effective to treat the disease. In a preferred embodiment, the disease is Parkinson's disease. In one embodiment of the subject invention, the treatment of Parkinsonian patients is long-term. The therapeutically effective amount of LDEE is preferably an amount from 0.1-1000 mg equivalent of levodopa.

In one embodiment, the subject invention provides a method of inducing in a human subject a therapeutically effective blood plasma level of levodopa comprising administering to the human subject

a controlled release formulation of levodopa ethyl ester, and

an immediate release formulation of levodopa ethyl ester,

wherein the therapeutically effective blood plasma level is at least 1000 ng of levodopa per ml of blood plasma within 50 minutes after the administration and no less than 100 ng of levodopa per ml of blood plasma at 6 hours after the administration.

In another embodiment, the therapeutically effective blood plasma level is at least 1250 ng of levodopa per ml of blood plasma within 50 minutes after the administration and no less than 110 ng of levodopa per ml of blood plasma at 6 hours after the administration.

In an additional embodiment, the therapeutically effective blood plasma level is at least 1500 ng of levodopa per ml of blood plasma within 50 minutes after the administration and no less than 120 ng of levodopa per ml of blood plasma at 6 hours after the administration.

In a further embodiment, the therapeutically effective blood plasma level is at least 1700 ng of levodopa per ml of blood plasma within 50 minutes after the administration and no less than 130 ng of levodopa per ml of blood plasma at 6 hours after the administration.

In still another embodiment, the therapeutically effective blood plasma level is at least 1850 ng of levodopa per ml of blood plasma within 50 minutes after the administration and no less than 140 ng of levodopa per ml of blood plasma at 6 hours after the administration.

In one embodiment, the therapeutically effective blood plasma level is at least 1900 ng of levodopa per ml of blood plasma within 50 minutes after the administration and no less than 150 ng of levodopa per ml of blood plasma at 6 hours after the administration.

In a further embodiment, the subject invention provides a method of inducing in a human subject a therapeutically effective blood plasma level of levodopa and carbidopa comprising administering to the human subject

a controlled release formulation of levodopa ethyl ester, and

an immediate release formulation of levodopa ethyl ester, in admixture with carbidopa,

wherein the therapeutically effective blood plasma level is at least 1000 ng of levodopa and 100 ng of carbidopa per ml of blood plasma within 50 minutes after the administration and no less than 100 ng of levodopa and 40 ng of carbidopa per ml of blood plasma at 6 hours after the administration.

In another embodiment, the therapeutically effective blood plasma level is at least 1250 ng of levodopa and 100 ng of carbidopa per ml of blood plasma within 50 minutes after the administration and no less than 110 ng of levodopa and 42 ng of carbidopa per ml of blood plasma at 6 hours after the administration.

In an additional embodiment, the therapeutically effective blood plasma level is at least 1500 ng of levodopa and 100 ng of carbidopa per ml of blood plasma within 50 minutes after the administration and no less than 120 ng of levodopa and 44 ng of carbidopa per ml of blood plasma at 6 hours after the administration.

In a further embodiment, the therapeutically effective blood plasma level is at least 1700 ng of levodopa and 100 ng of carbidopa per ml of blood plasma within 50 minutes after the administration and no less than 130 ng of levodopa and 46 ng of carbidopa per ml of blood plasma at 6 hours after the administration.

In another embodiment, the therapeutically effective blood plasma level is at least 1850 ng of levodopa and 100 ng of carbidopa per ml of blood plasma within 50 minutes after the administration and no less than 140 ng of levodopa and 48 ng of carbidopa per ml of blood plasma at 6 hours after the administration.

In another embodiment, the therapeutically effective blood plasma level is at least 1900 ng of levodopa and 100 ng of carbidopa per ml of blood plasma within 50 minutes after the administration and no less than 150 ng of levodopa and 50 ng of carbidopa per ml of blood plasma at 6 hours after the administration.

As discussed in the background, levodopa is often administered with a decarboxylase inhibitor. In a solid formulation, it is important that the rate of dissolution, and hence, the blood level, of the decarboxylase inhibitor be appropriate for that of the L-DOPA. In both the immediate release and the slow release formulations of carbidopa and L-DOPA, these two active ingredients are released at the same ratio. This release can be readily achieved in a matrix system because the chemical and physical properties of carbidopa and levodopa are similar. In monolithic matrix systems, the active agents are homogeneously dissolved or dispersed throughout a polymer mass or other carrier material. Release characteristics depend on the geometry of the system, the nature of the polymer and other excipients, solubility and the processing methods. As the two active materials, carbidopa and L-DOPA, were compressed under the same conditions, it was found that the in vitro release is a direct function of their solubility in the dissolution fluid. Both compounds are slightly soluble in water, degrade rapidly in alkaline media, and have similar solubility versus pH profiles, as well as similar solubility versus temperature profiles.

However, carbidopa and LDEE, examples of two active materials in the composition of the present invention, have different chemical and physical properties and contrasting solubility characteristics especially in acid conditions, i.e., in the gastrointestinal tract (see Table 1).

TABLE 1

| | Solubility at ˜22-24° C. (mg/ml) | | |
|---|---|---|---|
| pH | Levodopa | Carbidopa | LDEE |
| 1.0 | 5.38 | 21.4 | 2200 |
| 5.0 | 4.96 | 1.66 | 1700 |

Formulating a pharmaceutical composition from LDEE and carbidopa, examples of two active ingredients of the subject invention having different solubility characteristics, which were to be released with similar immediate and controlled release dissolution profiles from a dual release tablet presented a problem. In detail,

(a) Carbidopa contains 7.5% water whereas LDEE is highly sensitive to moisture, undergoes hydrolysis easily, and therefore would not be expected to be a candidate for a slow release formulation;

(b) LDEE has a tendency to act as a binder (it sticks).

(c) LDEE is not stable at room temperature and is kept under refrigeration (2-8° C.), whereas any tablet formulation must be designed, for optimum convenience to patients, pharmacists and physicians, for storage at room temperature.

The subject invention has overcome these difficulties. To attain optimal bioavailability of levodopa (from levodopa ethyl ester), the disclosed formulation uses carbidopa only in the immediate release portion of the tablet, while it employs levodopa ethyl ester in both the immediate release and the controlled release portions. The outer layer of the tablet comprises the fast onset “burst” immediate-release formulation. The internal core comprises a controlled or slow-release (up to 10 hours) formulation using an approved cellulose derivative which swells and/or becomes gellable and erodible on contact with water or aqueous solutions. Tables 12 and 14 shows that the inner core exhibits controlled release of levodopa ethyl ester. Table 16 depicts the immediate release of levodopa ethyl ester and carbidopa from the outer layer. Carbidopa has a significantly higher half-life and lower solubility in the body than levodopa, so the immediate release carbidopa is present in the body longer than the levodopa metabolized from the immediate release levodopa ethyl ester. The levodopa ethyl ester in the controlled release layer continues to provide levodopa ethyl ester while therapeutic carbidopa levels remain in the plasma due to the longer half-life and lower solubility of the carbidopa. Thus, the disclosed tablet formulation provides therapeutically appropriate levels of levodopa ethyl ester and carbidopa in vivo.

As discussed in the background of the invention, Rubin (U.S. Pat. No. 6,238,699 B1) describes a formulation of carbidopa and levodopa in immediate and controlled release compartments. However, Rubin does not suggest the replacement of levodopa with LDEE or how to account for the difference in properties between the LDEE and levodopa. Given the differences in bio-availability and solubility described above, one skilled in the art would not be motivated to replace levodopa with LDEE to create a pharmaceutical composition containing LDEE and carbidopa in immediate and controlled release portions of a tablet as in the subject invention.

Chiesi et al. (WO 99/17745), also described in the background of the invention, disclose a three-layer monolithic system of levodopa methyl ester and carbidopa. Chiesi et al. show that the release of levodopa methyl ester is approximately concomitant with the release of carbidopa in a formulation in which levodopa methyl ester, but not carbidopa, is present in the slow release layer, while both levodopa methyl ester and carbidopa are used in the remaining layers. Additionally, the release profile of the levodopa methyl ester and the carbidopa in the compositions of Chiesi et al. is dependent upon the interactions between the layers of the composition.

In contrast to Chiesi et al., due to the different geometry of the pharmaceutical compositions of the subject invention, the release profiles of the LDEE and the carbidopa are independent. In the subject invention, the total release kinetic is the sum of the individual contributions and one can predict from the dissolution profile of the inner core the total release kinetics of the dual release tablet. Furthermore, the metabolic products of levodopa methyl ester are levodopa and methanol (which is toxic, see U.S. Pat. No. 5,354,885), while the metabolic products of LDEE are levodopa and ethanol. In addition, the tablet of the subject invention differs from Chiesi et al. in that the subject invention is easier to manufacture, is physically smaller, and is therefore expected to have higher patient compliance.

The LDEE used in the compositions of the present invention is preferably that as described in U.S. Pat. Nos. 6,218,566 or 5,525,631, the contents of which are hereby incorporated by reference. LDEE may be prepared following the procedure of U.S. Pat. Nos. 6,218,566; 5,607,969; 5,525,631; or 5,354,885; all of which are hereby incorporated by reference. Preferably, the LDEE is highly purified, non-hygroscopic and crystalline.

The subject invention provides press-coated tablets, multi-layered tablets and a combination of a matrix and a disintegrant tablet.

This invention will be better understood from the Experimental Details which follow. However, one skilled in the art will readily appreciate that the specific methods and results discussed are merely illustrative of the invention as described more fully in the claims which follow thereafter.

Experimental Details

LDEE was prepared as described in U.S. Pat. No. 6,218,566 B1. However, any pharmaceutically acceptable salt of LDEE can be used.

### Example 1

### Manufacture of LDEE Tablets

Inner Core

Inner core A (controlled release) was manufactured as follows:

Procedure A

An inner core was prepared by mixing LDEE with a carrier and several excipients (Table 2).

TABLE 2
Composition of inner core - controlled release

| Excipient | Use | A (mg/tablet) |
|---|---|---|
| L-Dopa ethyl ester | Active | 114 |
| | material | |
| Hydroxypropylmethyl- | Carrier | 50 |
| cellulose | | |
| Microcrystalline cellulose | Filler | 85 |
| Syloid ® | Flow agent | 3 |
| Sodium stearyl fumarate | Lubricant | 5 |
| Magnesium stearate | Lubricant | 3 |

Outer Layer

Procedure B

Outer layer B, the immediate release formulation, was prepared by granulating carbidopa monohydrate with a binding agent and several excipients (Table 3) and then mixing the carbidopa monohydrate granulate with LDEE and several excipients (Table 4).

TABLE 3
Composition of granulate for outer layer - immediate
release

| Excipient | Use | B (mg/tablet) |
|---|---|---|
| Carbidopa | Active material | 54 |
| monohydrate | | |
| Microcrystalline | Filler | 40 |
| cellulose | | |
| Starch 1500 ® | Disintegrating | 34 |
| | agent | |
| Hydroxypropyl- | Binding agent | 12 |
| cellulose | | |

TABLE 4
Outer layer

| | | B |
|---|---|---|
| Excipient | Use | (mg/tablet) |
| Granulate carbidopa | Active | 140 |
| monohydrate (see | material | |
| Table 3) | | |
| L-Dopa ethyl ester | Active | 114 |
| | material | |
| Microcrystalline | Filler | 32 |
| cellulose | | |
| Starch 1500 ® | Disintegrating | 4 |
| | agent | |
| Syloid ® | Flow agent | 3 |
| Sodium stearyl | Lubricant | 7.5 |
| fumarate | | |

Outer layer B was compressed onto inner core A to produce a press coated tablet.

### Example 2a

Each of the following inner cores K-I, K, L and M (prepared according to Procedure A) contained a different amount of carrier as described in Table 5 in order to determine the effect of the amount of the carrier on the dissolution rate (see Table 6).

TABLE 5
Variation in the amount of carrier

| | | | K-I | K | L | M |
|---|---|---|---|---|---|---|
| | Excipient | Use | mg/tab | mg/tab | mg/tab* | mg/tab |
| Inner | L-Dopa ethyl | Active | 114 | 114 | 114 | 114 |
| Core | ester | material | | | | |
| | Hydroxypropyl- | Carrier | 140 | 70 | 50 | 35 |
| | methylcellulose | | | | | |
| | Microcrystalline | Filler | 0 | 65 | 85 | 100 |
| | cellulose | | | | | |
| | Syloid ® | Flow | 3 | 3 | 3 | 3 |
| | | agent | | | | |
| | Sodium stearyl | Lubri- | 5 | 5 | 5 | 5 |
| | fumarate | cant | | | | |
| | Magnesium | Lubri- | 2.5 | 2.5 | 2.5 | 0 |
| | stearate | cant | | | | |

*Inner core L is identical to inner core A (described in Example 1)

Each inner core formulation was then tested in a dissolution test using 900 ml 0.1 N HCl at 37° C. in US Pharmacopeia (USP) Apparatus 2 at 75 RPM.

The release profile for each inner core formulation over an 8 hour period is shown in Table 6.

TABLE 6
Dissolution of LDEE of inner cores K-I, K, L and M

| | K-I | K | L | M |
|---|---|---|---|---|
| Time | % Release | % Release | % Release | % Release |
| (hour) | LDEE | LDEE | LDEE | LDEE |
| 0.5 | 17 | 22 | 31 | 61 |
| 1 | 26 | 34 | 47 | 75 |
| 1.5 | | | 58 | 83 |
| 2 | 40 | 52 | 67 | 87 |
| 2.5 | | | 75 | 90 |
| 3 | | | 83 | 92 |
| 4 | 61 | 78 | | |
| 6 | 77 | 93 | | |
| 8 | 89 | 99 | | |

The dissolution profile was found to be dependent upon the amount of the carrier (Methocel® K100LV). For a dissolution profile of 8 hours, the amount of the carrier (Methocel® K100LV) was around 140 mg/tab (inner core K-I). For a dissolution profile of 4 hours, the amount of the carrier (Methocel® K100LV) was around 50 mg/tab (inner core L).

### Example 2b

Additional inner cores with different amounts of carrier were made according to Procedure A. The components of these inner cores are shown in Table 7.

TABLE 7
Inner core formulations containing different amounts of
carrier

| | | Ee | Ff | Gg |
|---|---|---|---|---|
| Excipient | Use | (mg/tab) | (mg/tab) | (mg/tab) |
| L-Dopa ethyl | Active | 152 | 152 | 152 |
| ester | material | | | |
| Methocel K100LV ® | Carrier | 50 | 45 | 40 |
| Microcrystalline | Filler | 44 | 49 | 54 |
| cellulose | | | | |
| Syloid ® | Flow agent | 2.7 | 2.7 | 2.7 |
| Sodium Stearyl | Lubricant | 5.0 | 5.0 | 5.0 |
| fumarate | | | | |
| Magnesium | Lubricant | 2.5 | 2.5 | 2.5 |
| stearate | | | | |

Table 8 portrays the effect of different amounts of carrier on dissolution rates of levodopa ethyl ester.

TABLE 8
Dissolution rates of LDEE in inner cores containing
different amounts of carrier

| | Ee | Ff | Gg |
|---|---|---|---|
| | (% Release | (% Release | (% Release |
| Time | of LDEE) | of LDEE) | of LDEE) |
| 30 | 30 | 58 | 69 |
| 60 | 46 | 77 | 93 |
| 90 | 58 | 89 | 99 |
| 120 | 68 | 95 | 100 |
| 150 | 77 | 97 | 100 |
| 180 | 84 | 98 | 100 |
| 210 | 90 | 99 | 100 |
| 240 | 95 | 99 | 100 |

As can be seen in Table 8, lowering the amount of the carrier resulted in higher dissolution rates of levodopa ethyl ester.

### Example 2c

Again, the amount of carrier in the inner core was varied in order to assess its effect on the dissoution rate of levodopa ethyl ester.

TABLE 9
Inner cores with varying amounts of carrier

| | | Hh | Ii | Jj |
|---|---|---|---|---|
| Excipient | Use | (mg/tab) | (mg/tab) | (mg/tab) |
| L-Dopa ethyl | Active | 182 | 182 | 182 |
| ester | material | | | |
| Methocel K100LV ® | Carrier | 48 | 43 | 40 |
| Microcrystalline | Filler | 20 | 25 | 23 |
| cellulose | | | | |
| Syloid ® | Flow agent | 2.7 | 2.7 | 2.7 |
| Sodium Stearyl | Lubricant | 5.0 | 5.0 | 5.0 |
| fumarate | | | | |
| Magnesium | Lubricant | 2.5 | 2.5 | 2.5 |
| stearate | | | | |

The differences in dissolution rate of inner cores comprising varying amounts of carrier are displayed in Table 10.

TABLE 10
Effect of varying amounts of carrier in inner core on
dissolution rate of levdopa ethyl ester

| | Hh | Ii | Jj |
|---|---|---|---|
| | (% Release | (% Release | (% Release |
| Time | of LDEE) | of LDEE) | of LDEE) |
| 30 | 46 | 61 | 73 |
| 60 | 68 | 82 | 93 |
| 90 | 80 | 91 | 99 |
| 120 | 88 | 96 | 101 |
| 150 | 93 | 98 | 102 |
| 180 | 96 | 98 | 102 |
| 210 | 98 | 98 | 102 |
| 240 | 99 | 98 | 101 |

Table 10 confirms the findings of Experiment 2a and 2b that lowering the amount of the carrier produced higher dissolution rates of levodopa ethyl ester. Thus, the carrier, Methocel KLOOLV®, functions as a slow release agent that retards the release of levodopa ethyl ester from the inner core.

### Example 3

Each one of inner cores Q and R (prepared according to Procedure A) contained different amounts of two lubricants to determine the optimal conditions for compression (see Table 11).

TABLE 11
Composition of the carrier

| | | | | R |
|---|---|---|---|---|
| | Excipient | Use | Q mg/tab* | mg/tab |
| Inner | L-Dopa ethyl ester | Active | 114 | 114 |
| core | | material | | |
| | Hydroxypropy methyl | Carrier | 50 | 50 |
| | cellulose | | | |
| | Microcrystalline | Filler | 85 | 85 |
| | cellulose | | | |
| | Syloid ® | Flow agent | 3 | 3 |
| | Sodium stearyl | Lubricant | 5 | 3 |
| | fumarate | | | |
| | Magnesium stearate | Lubricant | 3 | 6 |

*Inner core Q is identical to inner core A (described in Example 1)

The dissolution was performed as described in Example 2a. The release profile is shown in Table 12 below.

TABLE 12
Dissolution of LDEE of inner cores Q and R

| | Q | |
|---|---|---|
| Time | % Release | R |
| (hour) | LDEE | % Release LDEE |
| 0.5 | 31 | 30 |
| 1 | 47 | 45 |
| 1.5 | 58 | 56 |
| 2 | 67 | 66 |
| 2.5 | 75 | 75 |
| 3 | 83 | 83 |

Both inner cores Q and R gave good compression production. The rate of dissolution did not change with the varying amounts of the varying lubricants.

### Example 4

Inner cores containing different amounts of LDEE (Table 13) were prepared in order to determine the effect of the amount of LDEE on the dissolution profile (Table 14). Inner core ZB was prepared according to Procedure A, i.e., without pre-mixing the LDEE and a carrier, while inner cores ZC and ZCA were prepared by pre-mixing the LDEE with a carrier prior to being mixed with other excipients.

TABLE 13
Variation in the amount of LDEE

| | | | ZB | ZC | ZCA |
|---|---|---|---|---|---|
| | Excipient | Use | mg/tab* | mg/tab | mg/tab |
| Inner | L-Dopa ethyl | Active | 114 | 152 | 182 |
| core | ester | material | | | |
| | Methocel K100LV ® | Carrier | 50 | 50 | 48 |
| | (hydroxypropyl- | | | | |
| | methylcellulose) | | | | |
| | Microcrystalline | Filler | 85 | 44 | 20 |
| | cellulose | | | | |
| | Syloid ® | Flow | 2.7 | 2.7 | 2.7 |
| | | agent | | | |
| | Sodium stearyl | Lubricant | 5.0 | 5.0 | 5.0 |
| | fumarate | | | | |
| | Magnesium | Lubricant | 2.5 | 2.5 | 2.5 |
| | stearate | | | | |

*Inner core ZB is identical to inner core A (described in Example 1)

TABLE 14
Dissolution profile of LDEE

| | ZB | ZC | ZCA |
|---|---|---|---|
| | (% Release | (% Release | (% Release |
| Time | of LDEE) | of LDEE) | of LDEE) |
| 30 | 31 | 46 | 46 |
| 60 | 47 | 64 | 68 |
| 90 | 58 | 77 | 80 |
| 120 | 67 | 85 | 88 |
| 150 | 75 | 92 | 93 |
| 180 | 83 | 95 | 96 |
| 210 | 84 | 98 | 98 |
| 240 | 87 | 99 | 99 |

Increasing the amount of levodopa ethyl ester from 152 mg/tab to 182 mg/tab produced no significant differences in dissolution profile. However, significant differences in dissolution profile were obtained when the amount of levodopa ethyl ester was raised from 114 mg/tab to 152 mg/tab. This difference was a result of increasing the ratio between the levodopa ethyl ester/carrier from 2:1 (Inner core ZB) to 3:1 (Inner core ZC). This increase had a significant effect on the “wrapping” effect of the carrier on the active material. When the ratio of levodopa ethyl ester/carrier was 2:1, the amount of carrier was sufficient to provide the wrapping effect, resulting in a slower dissolution rate. In order to achieve a slower dissolution rate and a better effect of the carrier on the active material, the levodopa ethyl ester could be pre-mixed with the carrier, for example, in a ratio of 2:1 or 3:1 of levodopa ethyl ester relative to carrier.

### Example 5

The following tablet was prepared according to Procedure B, containing an immediate release formulation of LDEE and carbidopa monohydrate (“outer layer” of the subject invention without the “inner core”) (Table 15).

TABLE 15
Composition of outer layer

| | | | Amount |
|---|---|---|---|
| Outer layer | Excipient | Use | (mg/tab) |
| Carbidopa | Carbidopa | Active material | 54 |
| monohydrate | monohydrate | | |
| granulate | Microcrystaline | Filler | 40 |
| | cellulose | | |
| | Starch 1500 ® | Disintegrating agent | 34 |
| | Hydroxypropyl- | Binding agent | 12 |
| | cellulose | | |
| | L-DOPA ethyl ester | Active | 114 |
| | Microcrystaline | Filler | 32 |
| | cellulose | | |
| | Starch | Disintegrating Agent | 4 |
| | Sodium stearyl | Lubricant | 7.5 |
| | fumarate | | |

The dissolution of LDEE/carbidopa monohydrate from the immediate release formulation was tested in a dissolution test (USP, Apparatus 2, 75 rpm) as in Example 2a. The dissolution profile is presented in Table 16.

TABLE 16
Dissolution profile of outer layer

| Time (minutes) | Carbidopa monohydrate | LDEE |
|---|---|---|
| 5 | 97 | 88 |
| 10 | 97 | 89 |
| 15 | 97 | 90 |

In the immediate release formulation, the release of LDEE and carbidopa monohydrate is the same. The entire amount of the 2 active materials in the outer layer was released after 5 minutes. The inherent solubility of the actives cause immediate dissolution.

### Example 6

The following tablet was prepared (see Table 17) following the procedure of Example 1.

TABLE 17
Dual release tablet compositions

| | | | | X |
|---|---|---|---|---|
| | | | | (mg/ |
| Portion of Tablet | | Excipient | Use | tab) |
| Inner core | | L-Dopa ethyl ester | Active material | 114 |
| | | Hydroxypropyl- | Carrier | 70 |
| | | methylcellulose | | |
| | | Microcrystalline | Filler | 85 |
| | | cellulose | | |
| | | Syloid ® | Flow agent | 3 |
| | | Sodium stearyl | Lubricant | 5 |
| | | fumarate | | |
| | | Magnesium | Lubricant | 2.5 |
| | | stearate | | |
| | | Excipient | Use | |
| Outer | Carbidopa | Carbidopa monohydrate | Active material | 54 |
| layer | monohydrate | Microcrystalline | Filler | 40 |
| | granulate | cellulose | | |
| | | Starch 1500 ® | Disintegrating | 34 |
| | | | agent | |
| | | Hydroxypropyl- | Binding agent | 12 |
| | | cellulose | | |
| | | L-Dopa ethyl | Active material | 114 |
| | | ester | | |
| | | Microcrystalline | Filler | 32 |
| | | cellulose | | |
| | | Starch 1500 ® | Disintegrating | 4 |
| | | | agent | |
| | | Syloid ® | Flow agent | 3 |
| | | Sodium stearyl | Lubricant | 7.5 |
| | | fumarate | | |

The release profile was determined as in Example 2a and is shown in Table 18 below.

TABLE 18
Dissolution of LDEE/carbidopa monohydrate
of Tablet X

X

| | % Release L-Dopa | % Release |
|---|---|---|
| | ethyl | carbidopa |
| Time (min) | ester | monohydrate |
| 5 | 52 | 96 |
| 10 | 56 | 101 |
| 30 | 67 | 105 |
| 60 | 74 | 106 |
| 90 | 78 | 106 |
| 120 | 83 | 106 |
| 150 | 87 | 106 |
| 180 | 89 | 107 |

In tablet X, the entire amount of carbidopa monohydrate and 50% of LDEE was released after 5 minutes and the rest of the entire amount of LDEE was released in a controlled manner over 3 hours.

### Example 7

In one embodiment, the method of manufacturing the controlled release formulation of the inner core involves a step wherein prior to mixing the LDEE with the excipients, the LDEE is pre-mixed with the carrier in order to “wrap” the active material. The “wrapping” of the LDEE has the effect of providing the active ingredient with a slower dissolution rate (see Tables 19-22).

Inner cores were manufactured containing the amounts of components listed in Table 19. Inner core ZD was formed by mixing LDEE with the carrier (pre-mixing), and then the remaining excipients were mixed in. For comparison, inner cores ZE and ZF were manufactured according to Procedure A.

TABLE 19
Variations in the amount of the carrier and in the
manufacturing processes

| | | | ZD | ZE | ZF |
|---|---|---|---|---|---|
| | Excipient | Use | mg/tab | mg/tab | mg/tab |
| Inner | L-Dopa ethyl ester | Active | 152 | 152 | 152 |
| Core | | material | | | |
| | Methocel K100LV ® | Carrier | 57 | 54 | 50 |
| | Microcrystalline | Filler | 41 | 44 | 44 |
| | cellulose | | | | |
| | Syloid ® | Flow | 2.7 | 2.7 | 2.7 |
| | | agent | | | |
| | Sodium stearyl | Lubricant | 5.0 | 5.0 | 5 |
| | fumarate | | | | |
| | Magnesium stearate | Lubricant | 2.5 | 2.5 | 2.5 |

The inner cores were tested in a dissolution test according to Example 2a. The dissolution profiles are presented in Table 20.

TABLE 20
Dissolution of LDEE of inner cores ZD-ZF

| Time | ZD | ZE | ZF |
|---|---|---|---|
| (hours) | % Release LDEE | % Release LDEE | % Release LDEE |
| 0.5 | 36 | 43 | 42 |
| 1 | 51 | 61 | 60 |
| 1.5 | 60 | 72 | 72 |
| 2 | 69 | 80 | 80 |
| 2.5 | 75 | 85 | 86 |
| 3 | 81 | 90 | 91 |
| 3.5 | 86 | 93 | 95 |
| 4 | 90 | 96 | 97 |

As can be seen in Table 20, no significant differences in dissolution rate were obtained when the amount of carrier was increased, but the carrier was not pre-mixed with the LDEE (ZE and ZF formulations). The dissolution rate was slowed only by a combination of a pre-mix step and an increased amount of carrier (formulation ZD).

Additional inner cores were produced according to Table 21. During the manufacture of tablets Aa and Cc, the levodopa ethyl ester was pre-mixed with Methocel K100LV® prior to the addition of the other excipients. Tablets Bb and Dd were produced by mixing all components together without a premixing step.

TABLE 21
Tablet formulations (inner core) comprising different
amounts of carrier and different processes of manufacture

| | | Aa (mg/ | Bb (mg/ | Cc | Dd |
|---|---|---|---|---|---|
| Excipient | Use | tab) | tab) | (mg/tab) | (mg/tab) |
| L-Dopa ethyl | Active | 152 | 152 | 152 | 152 |
| ester | material | | | | |
| Methocel | Carrier | 50 | 50 | 40 | 40 |
| K100LV ® | | | | | |
| Microcrystalline | Filler | 44 | 44 | 54 | 54 |
| cellulose | | | | | |
| Syloid | Flow | 2.7 | 2.7 | 2.7 | 2.7 |
| | agent | | | | |
| Sodium | Lubricant | 5.0 | 5.0 | 5.0 | 5.0 |
| Stearyl | | | | | |
| fumarate | | | | | |
| Magnesium | Lubricant | 2.5 | 2.5 | 2.5 | 2.5 |
| stearate | | | | | |

The dissolution profiles shown in Table 22 were determined as in Example 2a.

TABLE 22
Effect on LDEE dissolution of different amounts of
carrier and premixing LDEE and a carrier

| | Aa | Bb | Cc | Dd |
|---|---|---|---|---|
| Time | (% Release | (% Release | (% Release | (% Release |
| (min) | of LDEE) | of LDEE) | of LDEE) | of LDEE) |
| 30 | 30 | 46 | 47 | 69 |
| 60 | 46 | 64 | 67 | 93 |
| 90 | 58 | 77 | 80 | 99 |
| 120 | 68 | 85 | 89 | 100 |
| 150 | 77 | 92 | 95 | 100 |
| 180 | 84 | 95 | 100 | 100 |
| 210 | 90 | 98 | 102 | 100 |
| 240 | 95 | 99 | 103 | 100 |

As can be seen in Table 22, significant differences in dissolution rate were observed when the LDEE was pre-mixed with the carrier.

### Example 8

The dual release tablet was formulated to be independent of the pH of the medium (see Tables 23-25). This feature is beneficial in designing an oral formulation of a drug for two reasons: a) In general, the pH of the stomach varies from patient to patient. Since rate of absorption of the drug is related to the pH of the stomach, a formulation that is pH independent should exhibit less variability among patients. Thus, such a formulation will have approximately equal bioavailability for all patients; b) In Parkinsons' patients, the action of the GI tract can be abnormal and can fluctuate during the course of the day, particularly pre- and post-prandially (GI dysmotility). A formulation that is not affected by pH will dissolve at a constant rate, independent of the time of day or whether the drug is taken before or after meals.

Thus, Tablet C was produced according to Table 23. First, the levodopa ethyl ester was mixed with Methocel K100LV®. Then, the mixture was compressed to form an inner core. Carbidopa monohydrate was then granulated with microcrystalline cellulose, Starch 1500®, and Klucel® to form a carbidopa monohydrate granulate. The carbidopa monohydrate granulate was then mixed with a second levodopa ethyl ester, microcrystalline cellulose, Starch 1500®, Syloid® and sodium stearyl fumarate. Then, this mixture was compressed to form an outer layer over the inner core, thereby manufacturing the tablet.

TABLE 23
Composition of dual release tablet C

| | | | | C (mg/ |
|---|---|---|---|---|
| Portion of Tablet | | Excipient | Use | tablet) |
| Inner Core | | L-Dopa ethyl ester | Active material | 152 |
| | | Methocel K100LV ® | Carrier | 57 |
| | | Microcrystalline | Filler | 41 |
| | | cellulose | | |
| | | Syloid ® | Flow agent | 2.7 |
| | | Magnesium stearate | Lubricant | 2.5 |
| | | Sodium Stearyl | Lubricant | 5 |
| | | fumarate | | |
| Outer | Carbidopa | Carbidopa | Active material | 54 |
| layer | monohydrate | monohydrate | | |
| | granulate | Microcrystalline | Filler | 40 |
| | | cellulose | | |
| | | starch 1500 ® | Disintegrating | 34 |
| | | | agent | |
| | | Klucel ® | Binding agent | 12 |
| | | L-Dopa ethyl ester | Active material | 76 |
| | | Microcrystalline | Filler | 170 |
| | | cellulose | | |
| | | Starch 1500 ® | Disintegrating | 4 |
| | | | agent | |
| | | Syloid ® | Flow agent | 3 |
| | | Sodium Stearyl | Lubricant | 7.5 |
| | | Fumarate | | |

The percent release of LDEE of Tablet C was tested following the procedure outlined in Example 2a except that the pH was adjusted as in Table 24.

TABLE 24
Effect of pH on release of LDEE
in tablet C (75 rpm)

| | pH | | |
|---|---|---|---|
| | 1.1 | 3 | 4.5 |
| Time (min) | % release LDEE | | |
| 10 | 43 | 53 | 52 |
| 30 | 55 | 65 | 63 |
| 60 | 67 | 76 | 73 |
| 90 | 74 | 84 | 80 |
| 120 | 80 | 89 | 84 |
| 150 | 85 | 91 | 88 |
| 180 | 88 | 93 | 91 |
| 210 | 91 | 95 | 92 |
| 240 | 94 | 96 | 94 |

For comparison, Table 25 shows the percent release of carbidopa in a commerical controlled release formulation, which was measured following Example 2a except for the variation in pH outlined in Table 25.

TABLE 25
Effect of pH dissolution medium on release profile of
levodopa/carbidopa monohydrate controlled release
tablet (Sinemet CR ®) (75 rpm)

| | pH | | |
|---|---|---|---|
| | 1.1 | 3 | 4.5 |
| Time | % Release carbidopa | | |
| (min) | monohydrate | | |
| 30 | 50 | 14 | 16 |
| 60 | 82 | 26 | 30 |
| 90 | 96 | 37 | 42 |
| 120 | 97 | 45 | 51 |
| 150 | 97 | 54 | 59 |
| 180 | 98 | 62 | 66 |
| 210 | 98 | 70 | 73 |
| 240 | 98 | 76 | 79 |

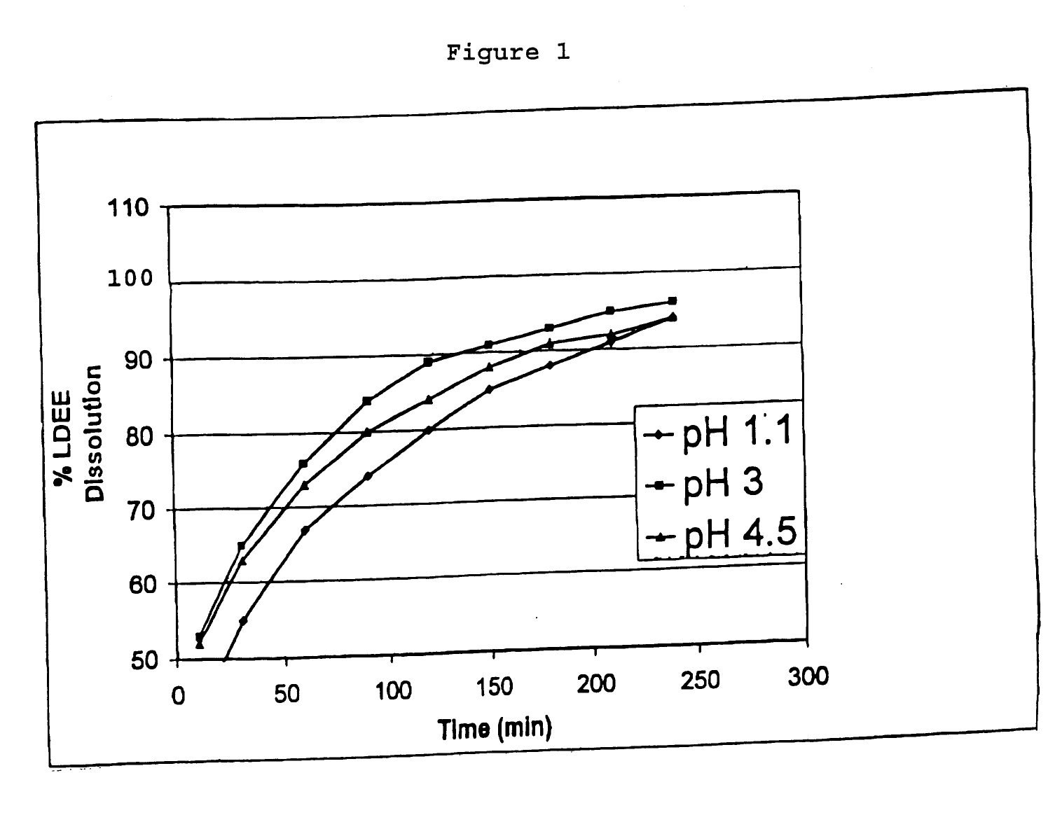

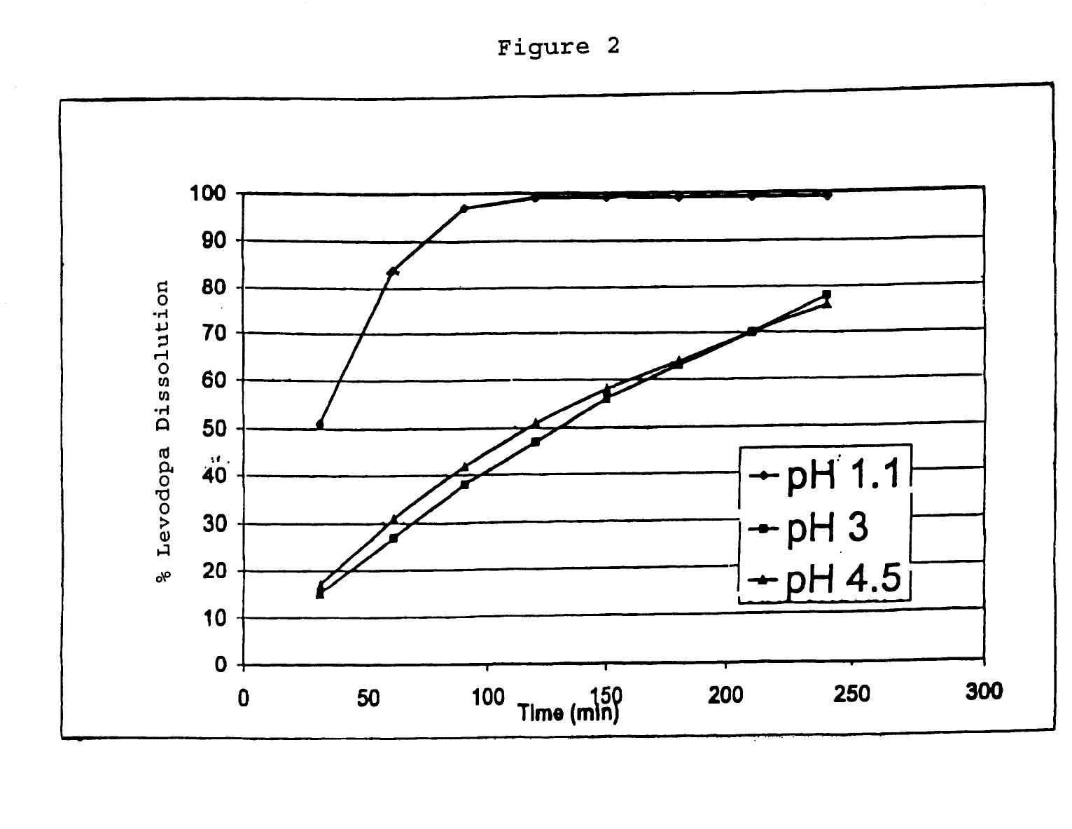

The data from Tables 24-25 is presented in graphical form in FIGS. 1-2. From these figures, it is evident that the LDEE formulation of the subject invention is independent of pH, while the commercial L-DOPA preparation is affected by variations in pH.

### Example 9

In order to test the correlation between the in-vivo and in vitro results, a bioavailability study in healthy volunteers (N=24) was carried out. Each volunteer received on an empty stomach one of the three formulations (tablets) described in Table 26 at a different period of time separated by a 7 day wash out and 2 day pre-treatment with carbidopa monohydrate, during which one dose of 25 mg of carbidopa monohydrate was administered each of the 2 days prior to the study. Blood samples were taken and plasma levels of carbidopa monohydrate and levodopa from the four treatments were monitored. The following formulations were prepared according to Table 26. The inner core of Formulation 1 was prepared according to Procedure A, i.e., without pre-mixing the LDEE and the carrier, while the inner cores of Formulations 2 and 3 were prepared by pre-mixing the LDEE with the carrier prior to mixing in the other excipients. The outer layers of Formulations 1-3 were prepared according to Procedure B, i.e., by granulating carbidopa monohydrate with a binding agent and at least one outer core excipient and then mixing the carbidopa granulate with LDEE and at least one outer core excipient.

TABLE 26
Composition of Formulations 1-3

| Portion of | | | | | Formu- | Formu- |
|---|---|---|---|---|---|---|
| tablet | | Excipient | Use | Formulation 1 | lation 2 | lation 3 |
| Inner core | | L-Dopa ethyl ester | Active | 114 | 114 | 152 |
| | | | material | | | |
| | | Hydroxypropyl- | Carrier | 50 | 140 | 57 |
| | | methylcellulose | | | | |
| | | Microcrystalline | Filler | 85 | — | 41 |
| | | cellulose (Avicel ® | | | | |
| | | PH 112) | | | | |
| | | Syloid ® | Flow agent | 2.7 | 2.7 | 2.7 |
| | | Sodium stearyl | Lubricant | 5.0 | 5.0 | 5.0 |
| | | fumarate | | | | |
| | | Magnesium stearate | Lubricant | 2.5 | 2.5 | 2.5 |
| Outer | Granulate | Carbidopa | Active | 54 | 54 | 54 |
| layer | | monohydrate | material | | | |
| | | Microcrystalline | Filler | 40 | 40 | 40 |
| | | cellulose (Avicel ® | | | | |
| | | PH 101) | | | | |
| | | Starch 1500 ® | Disinte- | 34 | 34 | 34 |
| | | | grating | | | |
| | | | agent | | | |
| | | Hydroxypropyl- | Binding | 12 | 12 | 12 |
| | | cellulose | agent | | | |
| | | L-Dopa ethyl ester | Active | 114 | 114 | 76 |
| | | | material | | | |
| | | Microcrystalline | Filler | 132 | 132 | 170 |
| | | cellulose (Avicel ® | | | | |
| | | PH 112) | | | | |
| | | Starch 1500 ® | Disinte- | 4.0 | 4.0 | 4.0 |
| | | | grating | | | |
| | | | agent | | | |
| | | Syloid ® | Flow agent | 3.0 | 3.0 | 3.0 |
| | | Sodium stearyl | Lubricant | 7.5 | 7.5 | 7.5 |
| | | fumarate | | | | |

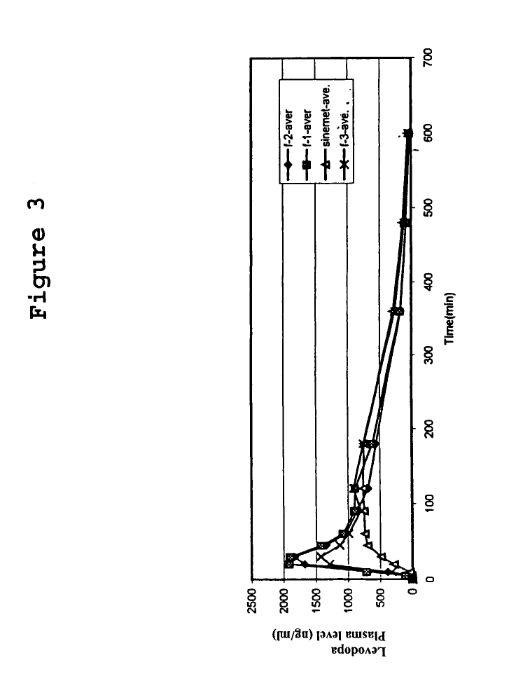

As can be seen in FIG. 3, the three formulations tested in the clinical trials display profiles of immediate and controlled release of LDEE. In addition, the bioavailability of the three formulations is higher within a short period of time after administration than that of Sinemet CR® (controlled release levodopa-carbidopa). The commercial product, Sinemet CR®, is shown as a reference in order to show the controlled release of levodopa in FIG. 3.

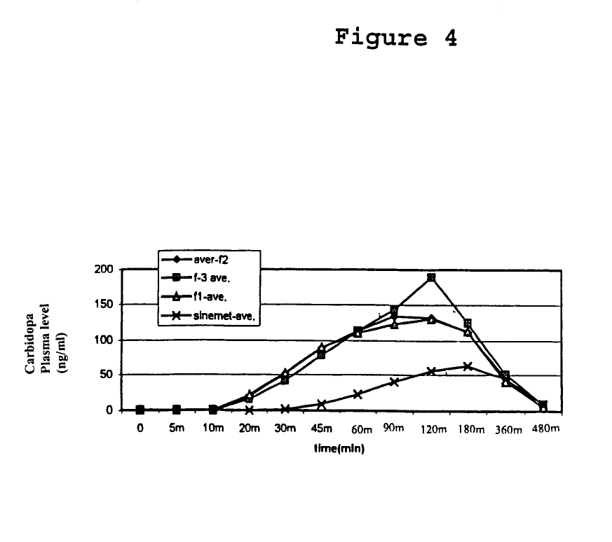

As can be seen in FIG. 4, the three formulations from the clinical trials display greater bioavailability of carbidopa than Sinemet CR@, which is used as a reference.

### Example 10

Additional tablets, Formulations 4-5, were prepared according to Table 27.

TABLE 27
Composition of Tablet Formulations 4-5

| Portion of | | | | | |
|---|---|---|---|---|---|
| Tablet | | Excipient | Use | Formulation 4 | Formulation 5 |
| Inner core | | L-Dopa ethyl | Active | 152 | 182 |
| | | ester | material | | |
| | | Hydroxypropyl- | Carrier | 40 | 40 |
| | | methyl | | | |
| | | cellulose | | | |
| | | Microcrystalline | Filler | 54 | 23 |
| | | cellulose | | | |
| | | (Avicel ® PH | | | |
| | | 112) | | | |
| | | Syloid | Flow | 2.7 | 2.7 |
| | | | agent | | |
| | | Sodium stearyl | Lubricant | 5.0 | 5.0 |
| | | fumarate | | | |
| | | Magnesium | Lubricant | 2.5 | 2.5 |
| | | stearate | | | |
| Outer | Granulate | Carbidopa | Active | 54 | 54 |
| layer | | monohydrate | material | | |
| | | Microcrystalline | Filler | 40 | 40 |
| | | cellulose | | | |
| | | (Avicel ® PH | | | |
| | | 101) | | | |
| | | Starch 1500 ® | Disintegrating | 34 | 34 |
| | | | agent | | |
| | | Hydroxypropyl- | Binding | 12 | 12 |
| | | cellulose | agent | | |
| | | L-Dopa ethyl | Active | 76 | 46 |
| | | ester | material | | |
| | | Microcrystalline | Filler | 170 | 200 |
| | | cellulose | | | |
| | | (Avicel ® PH | | | |
| | | 112) | | | |
| | | Starch 1500 ® | Disintegrating | 4.0 | 4.0 |
| | | | agent | | |
| | | Syloid ® | Flow | 3.0 | 3.0 |
| | | | agent | | |
| | | Sodium stearyl | Lubricant | 7.5 | 7.5 |
| | | fumarate | | | |

What is claimed is:

1. A tablet which comprises
an inner core formulated for controlled release consisting essentially of a mixture of levodopa ethyl ester or a derivative or a pharmaceutically acceptable salt thereof, a carrier and an inner core excipient component; and
an outer layer encapsulating the inner core and formulated for immediate release comprising a mixture of a decarboxylase inhibitor and levodopa ethyl ester or a derivative or a pharmaceutically acceptable salt thereof.

2. The tablet of claim 1, wherein in the inner core, the levodopa ethyl ester or derivative or pharmaceutically acceptable salt thereof is in a pre-mixture with the carrier.

3. The tablet of claim 1, wherein the decarboxylase inhibitor comprises carbidopa.

4. The tablet of claim 3, wherein the inner core formulated for controlled release consists essentially of a mixture of
(a) from about 4 mg up to about 400 mg levodopa ethyl ester or a derivative or a pharmaceutically acceptable salt thereof,
(b) a carrier, and
(c) an inner core excipient component; and
the outer layer encapsulating the inner core and formulated for immediate release comprises a mixture of:
(i) from above 0 mg up to about 200 mg carbidopa,
(ii) from about 5 mg up to about 300 mg levodopa ethyl ester or a derivative or a pharmaceutically acceptable salt thereof, and
(iii) an outer layer excipient component.

5. The tablet of claim 4, wherein components (a) and (b) are in a pre-mixture.

6. The tablet of claim 4 or 5, wherein in the outer layer, the carbidopa comprises granulated carbidopa.

7. The tablet of claim 4 or 5, wherein in the inner core, the levodopa ethyl ester or derivative or pharmaceutically acceptable salt thereof and the carrier are in a ratio of about 2:1 by weight.

8. The tablet of claim 4 or 5, wherein in the inner core, the carrier comprises a hydroxypropylmethylcellulose.

9. The tablet of claim 8, wherein the hydroxypropylmethylcellulose has an average molecular weight between about 10 kDa and about 1500 kDa.

10. The tablet of claim 9, wherein the hydroxypropylmethylcellulose has 19%-24% methoxyl substituent and 7%-12% hydroxylproproxyl substituent.

11. The tablet of claim 10, wherein the hydroxypropylmethylcellulose has a particle size distribution such that 65%-85% of the hydroxypropylmethylcellulose passes through a 100 mesh screen.

12. The tablet of claim 11, wherein the hydroxypropylmethylcellulose has a particle size distribution such that about 80% of the hydroxypropylmethylcellulose passes through a 100 mesh screen.

13. The tablet of claim 4 or 5, wherein the outer layer excipient component comprises an excipient used as a binding agent.

14. The tablet of claim 13, wherein the excipient used as a binding agent comprises a hydroxypropylcellulose.

15. The tablet of claim 4 or 5, wherein the outer layer excipient component comprises an excipient used as a disintegrating agent.

16. The tablet of claim 15, wherein the excipient used as a disintegrating agent comprises a starch.

17. The tablet of claim 16, wherein the starch is a partially pregelatinized maize starch.

18. The tablet of claim 4 or 5, wherein the inner core excipient component and the outer layer excipient component each comprise an excipient useful as a flow agent and/or an excipient useful as a lubricant.

19. The tablet of claim 18, wherein the excipient useful as a flow agent comprises a micron-sized silica powder.

20. The tablet of claim 18, wherein the excipient useful as a lubricant comprises magnesium stearate.

21. The tablet of claim 18, wherein the excipient useful as a lubricant comprises sodium stearyl fumarate.

22. The tablet of claim 18, wherein the inner core excipient component and the outer layer excipient component each comprise an excipient useful as a lubricant.

23. The tablet of claim 22, wherein the same excipient useful as a lubricant is present in both the inner core excipient component and the outer layer excipient component.

24. The tablet of claim 22 or 23, wherein the excipient useful as a lubricant present in the outer core excipient component comprises sodium stearyl fumarate.

25. The tablet of claim 22, wherein the excipient useful as a lubricant present in the inner core excipient component comprises sodium stearyl fumarate.

26. The tablet of claim 18, wherein the inner core excipient component comprises a first excipient useful as a lubricant and a second excipient useful as a lubricant.

27. The tablet of claim 26, wherein the first excipient usesful as a lubricant is sodium stearyl fumarate and the second excipient useful as a lubricant is magnesium stearate.

28. The tablet of claim 4 or 5, wherein the inner core excipient component and/or the outer layer excipient component comprises an excipient useful as a filler.

29. The tablet of claim 28, wherein the excipient useful as a filler comprises a microcrystalline cellulose.

30. The tablet of claim 29, wherein the microcrystalline cellulose has an average particle size between about 50 and about 90 microns.

31. The tablet of claim 6, wherein in the inner core,
the levodopa ethyl ester or derivative or pharmaceutically acceptable salt thereof in (a) is present in an amount from about 10 mg up to about 400 mg; and wherein in the outer layer,
the granulated carbidopa in (i) comprises from above 0 mg to about 75 mg carbidopa, and
the levodopa ethyl ester or derivative or pharmaceutically acceptable salt thereof in (ii) is present in an amount from about 10 mg up to about 250 mg.

32. The tablet of claim 31, wherein in the inner core
the levodopa ethyl ester or derivative or pharmaceutically acceptable salt thereof is present in an amount from about 50 mg up to about 400 mg;
and wherein in the outer layer
the granulated carbidopa comprises from about 10 mg up to about 50 mg carbidopa, and
the amount of the levodopa ethyl ester or the derivative or pharmaceutically acceptable salt thereof is from about 50 mg up to about 200 mg.

33. The tablet of claim 31, wherein in the inner core
the levodopa ethyl ester or derivative or pharmaceutically acceptable salt thereof is present in an amount from about 19 mg up to about 228 mg;
and wherein in the outer layer
the granulated carbidopa comprises from about 4.2 mg up to about 75 mg carbidopa, and
the levodopa ethyl ester or derivative or pharmaceutically acceptable salt thereof is present in an amount from about 19 mg up to about 228 mg.

34. The tablet of claim 4 or 5, wherein above 5% of the total levodopa ethyl ester or derivative or pharmaceutically acceptable salt thereof present in the tablet is in the outer layer.

35. The tablet of claim 34, wherein above 10% of the total levodopa ethyl ester or derivative or pharmaceutically acceptable salt thereof present in the tablet is in the outer layer.

36. The tablet of claim 35, wherein above 30% of the total levodopa ethyl ester or derivative or pharmaceutically acceptable salt thereof present in the tablet is in the outer layer.

37. The tablet of claim 36, wherein above 50% of the total levodopa ethyl ester or derivative or pharmaceutically acceptable salt thereof present in the tablet is in the outer layer.

38. The tablet of claim 37, wherein above 70% of the total levodopa ethyl ester or derivative or pharmaceutically acceptable salt thereof present in the tablet is in the outer layer.

39. The tablet of claim 4 or 5, wherein the total tablet comprises about 228.0 mg levodopa ethyl ester or derivative or pharmaceutically acceptable salt thereof, and about 50.0 mg carbidopa.

40. The tablet of claim 4 or 5, wherein the total tablet comprises about 114.0 mg levodopa ethyl ester or derivative or pharmaceutically acceptable salt thereof, and about 25.0 mg carbidopa.

41. The tablet of claim 4 or 5, wherein the total tablet comprises about 57.0 mg levodopa ethyl ester or derivative or pharmaceutically acceptable salt thereof, and about 12.5 mg carbidopa.

42. The tablet of claim 6, wherein in the inner core
the 4 mg up to about 400 mg levodopa ethyl ester or derivative or pharmaceutically acceptable salt thereof is present,
the carrier comprises from about 2.5 mg up to about 245 mg hydroxypropylmethylcellulose, and
the inner core excipient component comprises from 0 up to about 150 mg of a microcrystalline cellulose, from about 1 mg to about 10 mg of a micron-sized silica and from about 1 mg to about 30 mg sodium stearyl fumarate and about 1 mg to about 10 mg magnesium stearate;
and wherein in the outer layer
the granulated carbidopa is present in an amount from about 1 mg up to about 75 mg, and is present in a granulated admixture with 0 mg up to about 300 mg of a microcrystalline cellulose, from above 0 mg up to about 300 mg of a partially pregelatinized maize starch and from above 0 mg up to about 50 mg of a hydroxypropylcellulose,
from about 5 mg up to about 300 mg levodopa ethyl ester is present, and
the outer layer excipient component comprises above 0 mg up to about 300 mg of a microcrystalline cellulose, above 0 up to about 300 mg of a partially pregelatinized maize starch, above 0 mg up to about 50 mg of a micron-sized silica and from above 0 up to about 30 mg sodium stearyl fumarate.

43. The tablet of claim 42, wherein in the inner core
about 114 mg levodopa ethyl ester is present,
the carrier comprises about 50 mg of the hydroxypropylmethylcellulose, and
the inner core excipient component comprises about 85 mg of a microcrystalline cellulose, about 2.7 mg of a micron-sized silica, about 5 mg of sodium stearyl fumarate and about 2.5 mg magnesium stearate;
and wherein in the outer layer
about 54 mg granulated carbidopa is present in a granulated admixture with about 40 mg of a microcrystalline cellulose, about 34 mg of a partially pregelatinized maize starch, and about 12 mg of a hydroxypropyl cellulose,
about 114 mg levodopa ethyl ester is present, and
the outer layer excipient component comprises about 132 mg of a microcrystalline cellulose, about 4 mg of a partially pregelatinized maize starch, about 3 mg of a micron-sized silica, and about 7.5 mg sodium stearyl fumarate.

44. The tablet of claim 42, wherein in the inner core
about 114 mg levodopa ethyl ester is present,
the carrier comprises about 140 mg of a hydroxypropylmethylcellulose, and
the inner core excipient component comprises about 2.7 mg of a micron-sized silica, about 5 mg of sodium stearyl fumarate and about 2.5 mg magnesium stearate;
and wherein in the outer layer
about 54 mg granulated carbidopa is present in a granulated admixture with about 40 mg of a microcrystalline cellulose, about 34 mg of a partially pregelatinized maize starch, and about 12 mg of a hydroxypropyl cellulose,
about 114 mg levodopa ethyl ester is present, and
the outer layer excipient component comprises about 132 mg of a microcrystalline cellulose, about 4 mg of a partially pregelatinized maize starch, about 3 mg of a micron-sized silica, and about 7.5 mg sodium stearyl fumarate.

45. The tablet of claim 42, wherein in the inner core
about 152 mg levodopa ethyl ester is present,
the carrier comprises about 57 mg hydroxypropylmethylcellulose, and
the inner core excipient component comprises about 41 mg of a microcrystalline cellulose, about 2.7 mg of a micron-sized silica, about 5 mg sodium stearyl fumarate and about 2.5 mg magnesium stearate;
and wherein in the outer layer
the granulated carbidopa is present in an amount of about 54 mg, and is present in a granulated admixture with about 40 mg of a microcrystalline cellulose, about 34 mg of a partially pregelatinized maize starch and about 12 mg of a hydroxypropylcellulose,
about 76 mg levodopa ethyl ester is present, and
the outer layer excipient component comprises about 170 mg of a microcrystalline cellulose, about 4 mg of a partially pregelatinized maize starch, about 3 mg of a micron-sized silica and about 7.5 mg sodium stearyl fumarate.

46. The tablet of claim 42, wherein in the inner core
about 152 mg levodopa ethyl ester is present,
the carrier comprises about 40 mg hydroxypropylmethylcellulose, and
the inner core excipient component comprises about 54 mg of a microcrystalline cellulose, about 2.7 mg of a micron-sized silica, about 5 mg sodium stearyl fumarate and about 2.5 mg magnesium stearate;
and wherein in the outer layer
the granulated carbidopa is present in an amount of about 54 mg, and is present in a granulated admixture with about 40 mg of a microcrystalline cellulose, about 34 mg of a partially pregelatinized maize starch and about 12 mg of a hydroxypropylcellulose,
about 76 mg levodopa ethyl ester is present, and
the outer layer excipient component comprises about 170 mg of a microcrystalline cellulose, about 4 mg of a partially pregelatinized maize starch, about 3 mg of a micron-sized silica and about 7.5 mg sodium stearyl fumarate.

47. The tablet of claim 42, wherein in the inner core
about 182 mg levodopa ethyl ester is present,
the carrier comprises about 40 mg hydroxypropylmethylcellulose, and
the inner core excipient component comprises about 23 mg of a microcrystalline cellulose, about 2.7 mg of a micron-sized silica, about 5 mg sodium stearyl fumarate and about 2.5 mg magnesium stearate;
and wherein in the outer layer
the granulated carbidopa is present in an amount of about 54 mg, and is present in a granulated admixture with about 40 mg of a microcrystalline cellulose, about 34 mg of a partially pregelatinized maize starch and about 12 mg of a hydroxypropylcellulose,
about 46 mg levodopa ethyl ester is present, and
the outer layer excipient component comprises about 200 mg of a microcrystalline cellulose, about 4 mg of a partially pregelatinized maize starch, about 3 mg of a micron-sized silica and about 7.5 mg sodium stearyl fumarate.

46. A tablet which comprises
an inner core formulated for controlled release comprising
(a) a pre-mixture of levodopa ethyl ester or a derivative or a pharmaceutically acceptable salt thereof and a carrier, and
(b) at least one inner core excipient component; and
an outer layer encapsulating the inner core and formulated for immediate release comprising a mixture of carbidopa and levodopa ethyl ester or a derivative or a pharmaceutically acceptable salt thereof.

47. The tablet of claim 46, wherein in the inner core, the levodopa ethyl ester or derivative or pharmaceutically acceptable salt thereof and the carrier are in a ratio of about 2:1 by weight.

48. The tablet of claim 46, wherein in the inner core, the levodopa ethyl ester or derivative or pharmaceutically acceptable salt thereof and the carrier are in a ratio of about than 3:1 by weight.

49. A tablet which comprises
an inner core formulated for controlled release comprising a mixture of
(a) from about 4 mg up to about 400 mg levodopa ethyl ester or a derivative or a pharmaceutically acceptable salt thereof, and
(b) an inner core excipient component;
and an outer layer encapsulating the inner core and formulated for immediate release comprising a mixture of
(i) a granulated admixture of from about 1 mg up to about 75 mg carbidopa and at least one excipient; and
(ii) from about 5 mg up to about 300 mg levodopa ethyl ester or a derivative or a pharmaceutically acceptable salt thereof, and
(iii)an outer layer excipient component.

50. A process for manufacturing the tablet of claim 1, comprising
(A) mixing the levodopa ethyl ester or derivative or pharmaceutically acceptable salt thereof with the carrier and the inner core excipient component;
(B) compressing the mixture from step (A) to form the inner core;
(C) separately mixing the decarboxylase inhibitor with the levodopa ethyl ester or derivative or pharmaceutically acceptable salt thereof and an outer layer excipient component; and
(D) compressing the mixture of step (C) over the inner core formed in step (B) to form an outer layer encapsulating the inner core so as to thereby manufacture the tablet.

51. A process for manufacturing the tablet of claim 2, comprising
(A) preparing a pre-mixture of the levodopa ethyl ester or derivative or pharmaceutically acceptable salt thereof and the carrier;
(B) mixing the pre-mixture from step (A) with the inner core excipient component;
(C) compressing the mixture from step (B) to form an inner core;
(D) separately mixing the decarboxylase inhibitor with an outer layer excipient component and the levodopa ethyl ester or derivative or pharmaceutically acceptable salt thereof; and
(E) compressing the mixture of step (D) over the inner core formed in step (C) to form an outer layer encapsulating the inner core so as to thereby manufacture the tablet.

52. The process of claim 50 or 51, wherein in the outer layer, the decarboxylase inhibitor comprises carbidopa.

53. The process of claim 52, wherein the carbidopa comprises granulated carbidopa.

54. A process for manufacturing the tablet of claim 4, comprising
(A) mixing from about 4 mg up to about 400 mg levodopa ethyl ester or a derivative or a pharmaceutically acceptable salt thereof with a carrier and an inner core excipient component;
(B) compressing the mixture from step (A) to form the inner core;
(C) separately mixing above 0 mg up to about 75 mg carbidopa with about 5 mg up to about 300 mg levodopa ethyl ester or a derivative or a pharmaceutically acceptable salt thereof and an outer layer excipient component; and
(D) compressing the mixture of step (C) over the inner core formed in step (B) to form an outer layer encapsulating the inner core so as to thereby manufacture the tablet.

55. A process for manufacturing the tablet of claim 5, comprising
(A) mixing about 4 mg up to about 400 mg of levodopa ethyl ester or derivative or pharmaceutically acceptable salt thereof with a carrier;
(B) mixing an inner core excipient component with the mixture from step (A);
(C) compressing the mixture from step (B) to form the inner core;
(D) separately mixing 0 mg up to about 75 mg of carbidopa, about mg up to about 300 mg of levodopa ethyl ester or derivative or pharmaceutically acceptable salt thereof and an outer layer excipient component; and
(E) compressing the mixture of step (D) over the inner core formed in step (C) to form an outer layer encapsulating the inner core so as to thereby manufacture the tablet.

56. A process for manufacturing the tablet of claim 49, comprising
(A) mixing from about 4 mg up to about 400 mg levodopa ethyl ester or a derivative or a pharmaceutically acceptable salt thereof with an inner core excipient component;
(B) compressing the mixture from step (A) to form the inner core;
(C) separately granulating from about 1 mg up to about 75 mg carbidopa with at least one excipient;
(D) mixing the granulated admixture from step (C) with about 5 mg up to about 300 mg levodopa ethyl ester or a derivative or a pharmaceutically acceptable salt thereof and an outer layer excipient component;
(E) compressing the mixture of step (D) over the inner core formed in step (B) to form an outer layer encapsulating the inner core so as to thereby manufacture the tablet.

57. A method of treating a subject suffering from a disease selected from the group consisting of Parkinson's disease, senile dementia, dementia of the Alzheimer's type, a memory disorder, depression, hyperactive syndrome, an affective illness, a neurodegenerative disease, a neurotoxic injury, brain ischemia, a head trauma injury, a spinal trauma injury, schizophrenia, an attention deficit disorder, multiple sclerosis, withdrawal symptoms, epilepsy, convulsions and seizures, which comprises administering to the subject the tablet of any one of claims 1, 4, 5, 46 or 49 in an amount effective to treat the disease.

58. The method of claim 57, wherein the disease is Parkinson's disease.

59. A method of inducing in a human subject a therapeutically effective blood plasma level of levodopa comprising administering to the human subject
a controlled release formulation of levodopa ethyl ester, and
an immediate release formulation of levodopa ethyl ester, wherein the therapeutically effective blood plasma level is at least 1000 ng of levodopa per ml of blood plasma within 50 minutes after the administration and no less than 100 ng of levodopa per ml of blood plasma at 6 hours after the administration.

60. A method of inducing in a human subject a therapeutically effective blood plasma level of levodopa and carbidopa comprising administering to the human subject
a controlled release formulation of levodopa ethyl ester, and
an immediate release formulation of levodopa ethyl ester, in admixture with carbidopa,
wherein the therapeutically effective blood plasma level is at least 1000 ng of levodopa and 100 ng of carbidopa per ml of blood plasma within 50 minutes after the administration and no less than 100 ng of levodopa and 40 ng of carbidopa per ml of blood plasma at 6 hours after the administration.
